(19) United States
(12) Patent Application Publication
(10) Pub. No.: US 2026/0360559 A1
(43) Pub. Date: Nov. 12, 2026

(54) HEALTH MONITORING

(71) Applicant: Yu Life Ltd., London (GB)

(72) Inventors: John William RONAYNE, London (GB); Matthew Saul DOLTIS, London (GB); John HART, London (GB)

(73) Assignee: Yu Life Ltd., London (GB)

(21) Appl. No.: 19/674,243

(22) Filed: May 12, 2026

Related U.S. Application Data
(63) Continuation-in-part of application No. PCT/EP2025/062931, filed on May 12, 2025.

(51) Int. Cl. G16H 50/30; G16H 10/20; G16H 10/60; G16H 50/20; G16H 50/70

(52) U.S. Cl. CPC G16H 50/30; G16H 10/20; G16H 10/60; G16H 50/20; G16H 50/70

(57) ABSTRACT

A method of updating a user risk profile, the method comprising: receiving a user health profile, comprising a plurality of health data metrics; receiving a user risk profile, comprising plurality of risk metrics; receiving historical outcome data comprising correlated historical health data metrics and historical risk metrics; and generating, based on the user health profile, a subset of the plurality of health data metrics from the user health profile, determining, based on the historical outcome data, a correlation between any of the subset health metrics, updating, based on a determined correlation, a subset of the plurality of risk metrics. Also disclosed are methods of determining and updating a health profile of a user, as well as protecting user health data.

### RELATED APPLICATION

This application is a Continuation-in-Part (CIP) of PCT Patent Application No. PCT/EP2025/062931 having International filing date of May 12, 2025. The contents of the above applications are all incorporated by reference as if fully set forth herein in their entirety.

### FIELD AND BACKGROUND OF THE INVENTION

This invention relates to a health monitoring system and method. More particularly, there is disclosed a method of retrieving granular health information to build accurate user health profiles to assist with health risk forecasting and mitigating identified risks with interventions. Various ways of engaging users and methods of maintaining data integrity are also disclosed.

Digital health applications or ‘apps’ are well known and have provided new ways for individuals to track and optimise their health. For instance, such digital apps may provide questions to a user to determine their lifestyle and health habits. Some suggest new activities or challenges (i.e. an intervention) for them to complete based on provided information. As a user completes more of these activities or challenges, their general health improves as a consequence.

Digital health applications typically track and optimise general health by improving sleep, eating habits, and movement. In some cases, specialist health applications could target a particular set of health outcomes-such as those related to, for example, smoking, but there is a lack of monitoring of an entire spectrum of health outcomes in the digital health application landscape. Particularly, a lack of monitoring health outcomes not flagged by the user themselves.

Furthermore, questions and interventions have traditionally been determined by these apps in a static fashion. When determining an appropriate set of questions or interventions, apps may not take into account how the lifestyle or behaviours of an individual changes over time. Without a broad spectrum of specific health outcomes driving the selection of questions, there is also a lack of mapping of question responses to tangible information about specific conditions and an inability to intervene or interrogate on risk health outcomes that are not known to and identified by the user. Apps may not track the success or failure of an individual to complete proposed interventions, or they may not factor in these attempts when deciding on new questions or interventions to suggest. Furthermore, when determining appropriate questions or interventions, this determination may not take into account previously answered questions, or may not take a holistic view of a user's health. For instance, apps may focus solely on metrics relating to cardiovascular health when suggesting questions or interventions related to cardiovascular health. This may mean that the relationship between different aspects of health over time are overlooked by these apps, leading to inappropriate or ineffective questions or interventions being suggested to an end user.

More broadly, optimising the health of an individual may also have effects that go beyond that person, where associated incorporation of appropriately chosen questions and interventions may have an effect. For instance, underwriting services, such as health insurance companies, will determine an individual's risk of mortality based on a given category, such as age or gender. These risks are based on values measured across an entire population, which means that risks and therefore premiums determined in this fashion are not always appropriate for individuals who are significantly healthier or unhealthier than the norm, or they may not change for individuals over time. This in turn means that healthy individuals will pay more than is appropriate. In addition, as with the determination of questions or interventions, this forecasting is traditionally done on an aggregate basis, which does not consider individual health aspects or relationships between different health aspects. In turn, this means that forecasting of risk on the part of underwriting services is made less accurate.

Consequently, there is a clear need for improved forecasting of health outcomes, taking into account all aspects of an individual's health over time. There is also a related but similarly clear need to improve selection of questions and interventions, which also take into account varied aspects of user health over time.

### SUMMARY OF THE INVENTION

Aspects and embodiments of the present invention are set out in the appended claims. These and other aspects and embodiments of the invention are also described herein.

The systems and methods disclosed provide a means for improving health outcomes through collecting responses to health questionnaires and/or their feedback on attempted or successful interventions from users, logging these responses, generating a new set of questions or interventions based at least partially on historical questions or interventions, before providing the new set of questions or interventions to the user for them to respond to or attempt.

In one example, there is a method of updating a user risk profile for controlling a user health outcome, the method comprising: receiving a user health profile, comprising a plurality of health data metrics; receiving a user risk profile, comprising a plurality of risk metrics; receiving historical outcome data comprising correlated historical health data metrics and historical risk metrics; and generating, based on the user health profile, a subset of the plurality of health data metrics from the user health profile; determining, based on the historical outcome data, a correlation between any of the subset health metrics; and updating, based on a determined correlation, a subset of the plurality of risk metrics. Beneficially, the user health profiles disclosed herein may represent a user as a combination of health outcomes or diseases wherein the combination of health outcomes or diseases are some combination of health outcomes that the user presents or is at risk of and health outcomes for which the user is low risk. The questions and interventions may therefore be used to inform all the above factors including the health outcomes for which the user is considered low risk. Therefore, a more effective means for predicting and consequently improving an end user's health is provided.

Preferably, the method further comprises updating all of the plurality of risk metrics of the user risk profile.

Preferably, the method further comprises: receiving external data, and wherein determining a correlation is further based on the external data.

Preferably, the external data comprises health related population statistics.

Preferably, the method further comprises: validating, by a validation trial, the determination of a correlation, wherein the validation trial compares the determined correlation to pre-existing medical data; and upon detecting a difference between the determined correlation and the preexisting medical data, raising an error.

Preferably, the method further comprises: generating, by interpolating between a first health data metric and a second health data metric, a third health data metric; and appending the subset of the plurality of health data metrics with the third health data metric.

Preferably, the method further comprises providing, to a third party, the updated user risk profile, preferably further providing the user health profile.

Preferably, the method further comprises outputting the updated user risk profile to a user health system, wherein the user health system is configured to transmit the updated user risk profile to at least one of: a third party; a database; or a user interface, preferably on a user device.

In one example, there is a method of determining a health profile of a user for controlling a user health outcome, the method comprising: retrieving a first question from a database of health questions; providing the first question to a user interface; receiving from the user interface an input from a user indicating a first answer to the first question; updating the user health profile in dependence on the first answer; and retrieving a second question from the database; wherein retrieving the second question is dependent on a historical correlation between the second question and the first answer. By taking this approach, fewer questions and interventions need to be posed, which means that less data is required to accurately determine a holistic view of an end user's health, which is as a result more efficient and comprehensive even with sparser data.

Preferably, each question in the database of health questions is associated with at least one of a plurality of health topics, wherein the first and second question are associated with different health topics.

Preferably, retrieving the first and second question is performed by a machine learning model trained to determine a correlation between the second question and the first answer to the first question.

Preferably, retrieving the second question from the database is in further dependence on the updated user health profile.

Preferably, the method further comprises: providing the second question to a user interface; receiving from the user interface an input from a user indicating a second answer to the second question; updating the user health profile in dependence on the second answer; and retrieving a third question from the database; wherein retrieving the third question is dependent on a historical correlation between the third question and the second answer, preferably wherein the method is performed iteratively.

Preferably, user answers to each provided question are stored in a database as historical response data; and wherein retrieving the second question from the database is in further dependence on the historical response data.

Preferably, the user health profile comprises a risk score defining a probability of a given health outcome, wherein retrieving the second question from the database is in further dependence on the risk score.

Preferably, each question in the database of health questions is associated with a base weight value, wherein retrieving the second question from the database is in further dependence on a probability distribution calculated from the base weight values.

Preferably, the base weight value is multiplied by a seasonality factor of the question, wherein each question the database is associated with a date and the seasonality factor is based on a difference between a current date and the associated date.

Preferably, each question in the database of questions is associated with an eligibility value and wherein the method is configured to only retrieve a question with an eligibility value above a predetermined eligibility threshold.

Preferably, the eligibility values of each question in the database of health questions are calculated based on a plurality of trigger conditions.

Preferably, the user health profile is a first user health profile, the method further comprising a second user health profile.

Preferably, the first answer is given a quality metric and wherein when retrieving a second question from the database the first answer is given more weight relative to the first question, in proportion to the quality metric.

Preferably, the quality metric given to the first answer is based on a response time of the user between providing the first question and receiving an input from the user, preferably the quality metric given to the first answer increasing proportional to the response time.

Preferably, the method further comprises: providing to a user a recommendation of an action; wherein the step of providing to a user a recommendation occurs probabilistically depending on a historical susceptibility of a user to recommendations.

Preferably, the method further comprises: assigning an effectiveness value to the recommendation based on a user input after providing to a user the recommendation; and calculating a relapse risk of a user based on the historical susceptibility of a user to recommendations and the effectiveness value of the recommendation.

Preferably, the user health profile comprises a plurality of data points, the method further comprises: upon detection of an invalid data point of the user health profile, interpolating said data point from adjacent data points of the user health profile.

Preferably, the method further comprises outputting, to the database of questions, the updated user health profile to thereby update the database of questions based on the updated user health profile.

In one example, there is a method of updating a user health profile for controlling a user health outcome, the method comprising: providing a first question to a user; receiving from the user an input indicating a first answer to the first question; and either: a) repeating the method with a further question to a user, in the event an engagement metric exceeds an engagement threshold; or b) terminating the method. By providing and measuring an engagement metric, user interest is maintained and therefore more data of greater quality can be obtained over the course of a question/intervention set.

Preferably, the engagement metric is based on a response time of a user, determined as the time between providing the first question to the user and receiving an input from the user.

Preferably, the method further comprises: determining a delay period based on the first question and the response time; pausing the method for the duration of the delay period; and restarting the method upon the termination of the delay period.

Preferably, the engagement threshold increases, by a predetermined increment, each time the method is repeated.

Preferably, the engagement threshold increases in proportion to the response time of the user.

Preferably, the method further comprises determining a user reward based on the number of times the method has repeated; and rewarding the user.

Preferably, the method further comprises determining a user reward based on the engagement metric.

Preferably, the method further comprises determining a user reward based on a susceptibility of a user to a reward, wherein the susceptibility of a user to a reward is based on historical health outcomes of the user.

Preferably, at least one question or answer is provided by the user interacting with a chatbot.

Preferably, the method further comprises outputting, to a rewards system, the updated user health profile, the rewards system being configured to provide to a user a reward based on the updated user health profile.

Preferably, controlling the user health profile comprises predicting a user health outcome.

Preferably, predicting the user health profile comprises diagnosing a user health outcome.

Preferably, the method further comprising outputting the prediction of the user health profile.

Preferably, the prediction is outputted to a third party.

Preferably, the prediction is outputted to a user interface.

Preferably, the prediction is outputted to a database.

Preferably, controlling the user health profile comprises improving a user health outcome.

Preferably, improving a user health outcome comprises providing medical advice.

Preferably, improving a user health outcome comprises providing an intervention.

Preferably, providing an intervention comprises determining an intervention from a plurality of interventions and displaying to a user the intervention.

Controlling a user health outcome may in certain instances be understood to mean determining, advising or informing in respect of a user health outcome. The outcome may be a predicted, forecast or future outcome.

In one example, there is a method of protecting user health data on a database, the method comprising: receiving a request from a third party to provide a subset of data from the database; determining a permission level of the third party, wherein the permission level of the third party is predetermined by the user; determining a granularity level of the requested subset of data; and i) if the granularity level of the requested subset of data is lower than the permission level of the third party, providing the requested subset of data to the third party; or ii) if the granularity level of the requested subset of data is higher than the permission level of the third party, modifying the subset of the database to reduce the granularity level until the granularity level is lower than the permission level of the third party. By controlling the access provided to a third party in this fashion, users maintain a greater degree of autonomy and security over their personal data. This may also improve interoperability and data exchange security with third parties.

Preferably, modifying the data comprises: calculating at least one metadata value of the subset, the at least one metadata value being based on the subset of data and having a granularity level lower than the subset of data; and providing, to the third party, the at least one metadata value.

In one example, there is a system configured to carry out any method described above.

The invention may reside in any, some or all of the following features, in any appropriate order:
a first and second user health profile corresponding to a user
the first user health profile evolves with time based on answers to health outcome specific questions received by a user interface of a user device of the user
at least some of the specific health outcome questions are correlated
the second user health profile provides at least a probability of morbidity of the user which evolves over time
the answers given by the user have a temporal element
the temporal element is used to inform at least one of the first and second user profile,
the temporal element is used to provide forecasting of future health outcomes
the first user health profile is used to generate user-specific interventions
the user-specific interventions are provided as an output to the user interface
the temporal element and answers are used to inform a relapse probability
the relapse probability describes a risk of relapse in a predetermined time frame
the relapse probability is based in part on a determined susceptibility to a user-specific intervention
a relapse probability identifies a reduction in a risk of the user for a specific health outcome and forecasts a future increase in the risk of the user for the specific health outcome.

In one example, there is a method of updating a user risk profile for controlling a user health outcome, the method comprising: receiving a user health profile, comprising a plurality of health data metrics; receiving a user risk profile, comprising a plurality of risk metrics; selecting, based on the user health profile, a question from a plurality of questions; providing, the question to a user interface; receiving a response to the question; updating the user health profile based on the received response to the question; updating, based on a determined correlation, a subset of the plurality of risk metrics related to the health outcome.

Preferably, the plurality of questions are stored in a structured database ordered by question metadata, the question metadata comprising one or more of: a question-type indicator; weighting fields; positioning/eligibility fields; and a runtime topic-weight modifier.

Preferably, the method further comprises: selecting a second question based on the response to the question or based on the updated user health profile.

Preferably, the method further comprises filtering the plurality of questions for eligibility based on one or more of: a question-type indicator; weighting fields; positioning/eligibility fields; time to complete the question and a runtime topic-weight modifier.

Preferably, filtering is based on a cooldown criterion which assigns an interval based on question type or time taken to answer the question.

Preferably, a second question is selected by probabilistic selection or sampling of the plurality of questions.

Preferably, a second question is selected based on a four-factor weighting and normalisation mechanism.

Preferably, a 15-answer sliding window is used for response weighting for topic aggregated answer weights.

Preferably, selecting a question is based on paired-question sequencing or forced co-delivery via pairing logic.

Preferably, the health profile is refined through an active adaptive questioning loop.

In one example, there is a method of updating a user risk profile for controlling a user health outcome, the method comprising: receiving a user health profile, comprising a plurality of health data metrics; receiving a user risk profile, comprising a plurality of risk metrics; retrieving, based on the user health profile and the user risk profile, a pathway related to a health outcome, wherein the pathway comprises a plurality of questions; selecting, based on the user health profile, a question from the plurality of questions; receiving a response to the question; updating the user health profile based on the received response to the question; updating, based on a determined correlation, a subset of the plurality of risk metrics related to the health outcome.

Preferably, the pathway further comprises a plurality of interventions.

Preferably, the pathway evolves with the user health profile, wherein a change in user health profile results in a change in one or more of the questions or inventions in the pathway.

Preferably, an intervention is provided based on the updated user health profile.

Preferably an evolving sequence of interventions is provided where the sequence of interventions evolves based on a user reaction to an intervention in the sequence.

Preferably, the pathway evolves based on a user reaction to the invention.

Preferably, subsequent questions responses are used to assess user susceptibility to the intervention.

Preferably, evolution of the pathway is based on user burden, fatigue, recent activity or likelihood of competition.

Preferably, if a user completes a goal, the method may comprise escalating to a more demanding or more targeted goal.

Preferably, if the user does not complete a goal, the method may comprise de-escalating to provide easier advice, switch topics, or select a substitute goal.

Preferably, pathway content, sequencing rules, thresholds and topic weightings are changed through configuration rather than hard-coded application logic.

Preferably, different goals or interventions have different follow-up windows, wherein the method further comprises what to follow up on and the follow-up should happen.

It will be appreciated by the skilled reader that the system may comprise further components responsible for enabling associated functionalities of the disclosed components, such as statistical sampling, algorithmic processing, data validation, pattern recognition or predictive modelling. It will also be appreciated that the components may be joined in any combination or number, or that separate components may be run within separate systems that intercommunicate. The skilled reader will also appreciate that data may also follow a separate or alternate route through the system, or that processes enacted by the components comprised in the system may leverage parallelism to any degree.

There is additionally disclosed a method for improving health outcomes associated with a given end user, through providing questions and interventions based on prior responses. This method may comprise: 1) having a user respond to a questionnaire and attempt interventions related to the end-user's behaviour and potential health risks; 2) receiving a request containing the completed questionnaire and interventions attempted; 3) extracting data related to the end-user's behaviour and potential health risks; 4) selecting new questions based on the extracted data; 5) selecting new interventions based on the extracted data; 6) packaging the new questions and new interventions together; and 7) returning the new questions to the end user for them to complete.

Further aspects of the invention may comprise one or more of the following:

Dual user health profiles, the two sets of user health profiles defining the users within the system that are kept largely separate except at the intervention stage. For a given user, the profile in the first set is used to inform dynamic health questionnaires (DHQs) and the corresponding user profile in the second set is used to inform risk and interventions (with one informing the other at certain points). Preferably, the first set may be a real time profile that is evolving in the system. For example, where there may be an elevated number of responses indicating unhealthy behaviours in the diet category, informing the model to ask more questions in that category. Preferably, the second set may be one pertaining to what a response or set of response means to the probability of someone's probability of mortality of morbidity) wherein the second set may be aggregated or masked to share with third party systems. Beneficially, the user health profiles disclosed herein may represent a user as a combination of health outcomes or diseases wherein the combination of health outcomes or diseases are some combination of health outcomes that the user presents or is at risk of and health outcomes for which the user is low risk. The DHQs may be used to inform all the above factors including the health outcomes for which the user is low risk.

A method of retrieving granular health information to build accurate user health profiles. Preferably, the method is largely in the provision of DHQs but environment specific related factors, other health metrics. Interval setting and frequency of occurrence setting for DHQs. There may be a mapping, preferably, a learned mapping of DHQs, to inform which topics to provide. Preferably, the questions are further mapped to seasonality factors and environmental factors which provide granularity in maintaining of profiles, risk prediction and provision of DHQs. Preferably, the questions are constructed to learn different aspects of a user: to determine how informed a user is; to determine longevity of habits; to determine user receptivity to intervention; and to determine user health. Preferably, a machine learning algorithm that is trained on historical user responses determines correlations and causal links between health outcomes and responses to questions or interventions.

Steps, heart rate, location information and other real-time, passively collected health metrics, such as step counts, activity and inactivity patterns, sleep data, heart rate, app engagement patterns, and other biometric data from wearable devices may also inform the user health profiles and provision of questions.

A method of engaging users. The method preferably comprising the learning of user behaviour to understand how best to retrieve information from the user. Preferably, the method further comprises curation and presentation of user specific variety in question types. Preferably, presenting information is also adjusted according to the user. AI for user specific engagement and use of a chatbot may also be included as well as user-specific use of rewards and incentives, monitoring of user susceptibility to rewards and question types and provision based on monitoring. The method may further comprise the use of game mechanics and snapshots/instant overview, advice/tips, recommendations, roadmap for user engagement.
Maintenance of data integrity may include but not be limited to methods of adhering (and proving adherence) to existing standards of different authorities, detection of misuse, interpolation of missing data or interpolation of data based on learned similarities in question types and provision of information at the correct granularity to third parties.
Future risk forecasting and intervention including but not limited to improved risk calculations, relapse prediction and determination of susceptibility to intervention. The methods and systems for future risk forecasting and intervention preferably comprise a mapping of variables obtained from large-scale longitudinal medical datasets (e.g. national health registries, cohort studies that pair long-term lifestyle data with subsequent health outcomes) to user profiles, a mapping of data retrieved by the system to health outcome predictors/(risk) scoring, anomaly identification.
Machine learning (ML) may be used to determine risk.
Trails may be conducted in a peer-reviewed fashion and adhering to the same rigour as standard medical trials, e.g. A sample population of 1400+ volunteers may provide their data, and the models may be used to predict their health outcomes—and validate how accurate the model is.
Health surveys/wellbeing reports may also be used in risk forecasting. Preferably, also the use of health profiles to generate personalised intervention strategies, including advice, goal-setting, and benefits recommendations. Preferably, the methods for risk forecasting further comprise the adaptive learning of interventions, ensuring delivery at the right time and in a personalised manner to optimise health impact. Intervention pathways may make recommendations from user health profiles to available third-party services (e.g. EAP, benefit platforms, company-specific perks), delivered in a similarly personalised fashion. Interventions Preferably, incorporating modified in-app game mechanics, such as customised quests and challenges (e.g. walking or meditation), with dynamically adjusted incentives and recommendations based on the user's profile. The questions and corresponding responses may have associated temporal data. For example, a response may comprise an answer to the question and a date time stamp of the answer. The temporal data associated with the questions and answers may allow a temporal user profile to be constructed. It also enables a likelihood of relapse to be determined based on the temporal data by forecasting the historical temporal data obtained from the questions and corresponding responses into the future. This disclosure also provides computer programs and computer program products comprising software code adapted, when executed on a data processing apparatus, to perform any of the methods and/or for embodying any of the apparatus and system features described herein, including any or all of the component steps of any method.

The disclosure also provides a computer or computing system (including networked or distributed systems) having an operating system which supports a computer program for carrying out any of the methods described herein and/or for embodying any of the apparatus or system features described herein.

The disclosure also provides a computer readable media having stored thereon any one or more of the computer programs aforesaid.

The disclosure extends to methods, system and apparatus substantially as herein described and/or as illustrated with reference to the accompanying figures.

The disclosure also provides a signal embodying a computer program or a computer program product for carrying out any of the methods described herein, and/or for embodying any of the apparatus features described herein, a method of transmitting such a signal, and a computer product having an operating system which supports a computer program for carrying out the methods described herein and/or for embodying any of the apparatus features described herein.

Any feature in one aspect of the disclosure may be applied to other aspects of the invention, in any appropriate combination. In particular, method aspects may be applied to apparatus aspects, and vice versa. As used herein, means plus function features may be expressed alternatively in terms of their corresponding structure, such as a suitably programmed processor and associated memory. Furthermore, features implanted in hardware may generally be implemented in software, and vice versa. Any reference to software and hardware features herein should be construed accordingly.

It should be appreciated that particular combinations of the various features described and defined in any aspects of the disclosure can be implemented and/or supplied and/or used independently.

### BRIEF DESCRIPTION OF THE SEVERAL VIEWS OF THE DRAWING(S)

FIG. 1 shows a schematic diagram of a health monitoring system;

FIG. 2 shows a schematic diagram of the Processing Engine;

FIG. 3 demonstrates an example user interaction and data flow through the system;

FIG. 4 demonstrates an example data flow;

FIG. 5 shows an example data flow through a health system;

FIG. 6 shows an example data flow through the heath system;

FIG. 7 shows an example data flow;

FIG. 8a shows a schematic diagram of a system 800 for managing a user's health;

FIG. 8b shows system 900, similar to system 800, where like numerals have been used for like features;

FIG. 9 shows an aspect of a health profile to inform the dynamic health questionnaires as a plurality of meta-metrics 930a-z as described in respect of FIG. 8b;

FIG. 10 shows schematic of the process steps used by the models to determine a risk metric;

FIG. 11 shows a schematic of the interaction of system 1100 with external services, similar to the system 800,900 of FIGS. 8-9;

FIG. 12 shows a schematic diagram of the implementation of the AI driven modelling and/or underwriting of FIG. 10;

FIG. 13 shows a schematic diagram of a system 1300, similar to the system of system 800 and 900 of FIGS. 8a and 8b;

FIG. 14 shows a contour plot, demonstrating a relationship between reduction of risk for end users and/or a 3rd-party client;

FIG. 15 shows a table showing key differences between traditional risk forecasting approaches; and

FIG. 16 shows an example method of informing a pathway.

### DESCRIPTION OF SPECIFIC EMBODIMENTS OF THE INVENTION

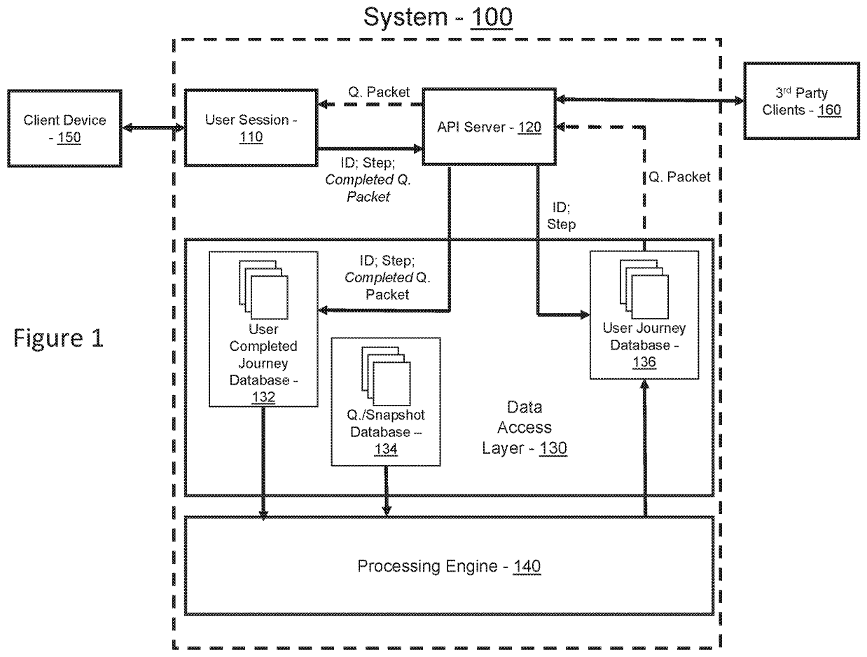

FIG. 1 shows a schematic diagram of a health monitoring system 100 for selecting questions and interventions based on previously completed questions and interventions. In the context of this disclosure, the term “completed questions or interventions” may also refer to questions and/or interventions that have at least been attempted by an end user, successfully or not, or questions or interventions that have been ignored by an end user. In either case, useful information related to a user's health can be determined from both successful and unsuccessful attempts. Similarly, “provided questions or interventions” may also refer to questions and/or interventions which have been selected by the system 100, but either have not been transmitted or acknowledged by an end user. The system 100 may be implemented as a web or phone application or accessed using such an application. Said web or phone application may be built using any suitable language, infrastructure or framework, such as FastAPI, Django, Flask, Angular, React, Vue.js, Express.js or Ruby on Rails.

In some embodiments, the system 100 comprises an API server 120, which communicates with a client device 150 associated with an end user, via a user session 110, said communication comprising data transfer between the client device 150 and API server 120. Specifically, API server 120 receives data from the client device 150 via the user session 110. The API server 120 further manages queries and mutations from the client device 150 and/or any 3rd-party client 160. Alternatively, the API server 120 may communicate directly with a client device 150, and vice versa, without need for a user session 110. The API server 120 is implemented in or communicates with a language or framework suitable for developing APIs, such as FastAPI or GraphQL. The API server 120 receives instructions originating from, for example, the client device or the 3rd-party client 160, for transmitting or manipulating (i.e. deleting, adding or otherwise modifying) data stored within the system, such as within or by the Data Access Layer 130.

Data transferred between the API server 120 and the client device 150 comprises one or more of:
data relating to completed questions or interventions, determined from responses provided to said questions or interventions provided by an end user on a client device 150, before being transferred to the API server 120 for use by the system 100;
data relating to newly determined questions or interventions, determined by the system 100 and sent to the client device 150 by the API server 120;
an ID associated with an end user or a specific client device 150;
personal data (e.g. gender; age; weight; geolocation data etc) which may be provided by a user;
a user ID associated with an end user, or with a client device 150, which is used to create a user session 110 associated with that user.

Transferred data is converted into data packets for transferal, which in turn comprise timestamps and checksums. Preferably, communication between the API server 120 and the client device 150 occurs in substantially real-time through a secure pipeline to prevent interception by non-authorised parties, provided an end user provides appropriate permissions for the system 100 to do so.

API server 120 additionally communicates with at least one 3rd-party client 160. The 3rd-party client 160 comprises web and mobile applications or databases related to, for example, insurance providers, employee benefit programs, biometric data services, wearable technologies, health monitoring technologies, wellbeing programmes, mental health subscriptions or sleep trackers, which may provide or receive data relating to an end user's health to the system. Communication between the API server 120 and the 3rd-party client 160 comprises data transfer between the two. Transferred data comprises any data suitable for determining a health profile of an end user, such as biometric data, or data that relates to services provided by the 3rd-party client 160, such as coverage policies and insurance risk profiles for an entire population. Transferred data for determining a health profile may comprise data determined by wearable technologies: heart rate (HR), the number of steps taken a day, maximal oxygen consumption or aerobic capacity (VO2), or metabolic equivalent of task (METs). Transferred data for determining a health profile may additionally comprise insurance risk profiles or metrics determined by insurance companies for the purposes of underwriting. Transferred data for determining a health profile is then transferred to the system 100 from the 3rd-party client 160 via the API server 120.

Data transferred between the API server 120 and the 3rd-party client 160 comprises questions or interventions either completed by or provided to an end user. Transferred data comprising questions or interventions is provided to the 3rd-party client 160 from the client device 150 via the API server 120. Transferred data originating from a database may then be sent to the 3rd-party client 160. Transferred data also comprise health risk profiles or health risk scores determined by the system 100 (such as by the Processing Engine 140, detailed below), which is sent to the 3rd-party client 160 via the API server 120. Alternatively, transferred data comprising questions or interventions may instead originate from a database stored on the system 100 (such as within the Data Access Layer 130, detailed below) or on an external server.

Data transferred between the API server 120 and the 3rd-party client 160 may take the form of data packets, which further comprise timestamps and checksums. Preferably, communication between the API server 120 and the 3rd-party client 160 occurs through a secure pipeline to prevent interception by non-authorised parties. Preferably, transferred data relating to personal information is provided at a sufficient level of granularity to prevent identification by the 3rd-party client 160. For example, transferred data relating to personal information may have names or geophysical locations removed or otherwise transformed to prevent identification. Alternatively, transferred data may aggregated or processed to remove identifying personal information before provision to the 3rd-party client 160.

The system 100 additionally comprises a Data Access Layer 130. The Data Access Layer 130 is responsible for loading and manipulating stored data related to an end user, comprising completed questions or interventions, or questions or interventions that an end user has been provided by the system 100. Said data may also comprise data related to an end user's ID, user session ID, health or risk profile, including any data received by the API server 120 from the 3rd-party client 160. For example, user health data may be personal data volunteered by an end user, such as the personal data provided from the client device 150 to the API server 120, as outlined above. As a further example, user health data may also comprise biometric data, such as the biometric data provided by the 3rd-party client 160 to the API server 120. As a yet further example, user health data may also comprise insurance risk profiles or metrics determined by an insurance provider, as outlined above. Alternatively, data provided to the Data Access Layer 130 may be aggregated across multiple end users before being stored.

User health data stored by the system 100 comprises a user risk profile, which in turn comprises many risk scores related to a given health topic to which questions and interventions may be assigned. An individual risk score may comprise a probability density function that describes the probability an individual develops an associated health outcome. As an example, a given health risk score may be assigned to a “depression” topic, which can then be associated with questions or interventions that assess an end user's feelings of depression. This “depression” topic may be associated with a broader “mental health” category, which in turn may be associated with other, larger health categories. A health risk profile may therefore comprise many health categories, forming a collection of scores associated with each topic. An overall score is then determined for an end user based on their associated health risk scores, signifying an overall level of health.

Data Access Layer 130 transfers any stored data to the API server 120, which then transfers the stored data to the client device 150 or the 3rd-party client 160. 3^{rd}-party client data, such as steps from health applications, are synced to and read by the API server 120. The Data Access Layer 130 includes operational datastores, which the API layer 120 reads and pushes to and reads from the client device 150. Data provided by the API server 120 to the Data Access Layer 130 is formatted into an appropriate format for storage by the Data Access Layer 130, such as JSON or YAML, before being incorporated into a database. Conversely, the Data Access Layer 130 may also load stored data from a database, before re-formatting it into a format appropriate for transmittance by the API server 120, such as data packets as outlined above. Alternatively, the Data Access Layer 130 may provide stored data directly to the API server 120, which may then re-format the data into a format appropriate for transmittance. The API server 120 may then in turn provide this data to the client device 150 or a 3rd-party client 160, as described above.

Data Access Layer 130 comprises at least one database, into which stored data is incorporated. The databases may be implemented using any suitable program for creating, storing or manipulating databases, such as MongoDB or PostgreSQL. As such, the Data Access Layer 130 may further process data in preparation for incorporation into a database, such as formatting strings and conversion of data into a suitable format, such as JSON or YAML. At least one database comprises a User Completed Journey Database (UCJD) 132, which further comprises data relating to questions or interventions completed by an end user. The UCJD 132 additionally comprises data related to a user's health, which further comprises biometric data, personal data, health risk profiles or insurance data, as outlined above. The Data Access Layer 130 also comprises a User Journey Database (UJD) 136, which stores questions or interventions that have been selected by the Processing Engine 140 and provided to an end user for completion, based on an end user's health profile as well as completed questions or interventions. Preferably, the Data Access Layer 130 may yet further comprise a Question and Intervention Database (QID) 134, which comprises questions or interventions that may potentially be selected by a Processing Engine 140 for provision to an end user. Still preferably, the Data Access Layer 130 may load requested data into a suitable in-memory structure, such as Pandas DataFrames, as when the system 100, is started by an end user. Alternatively, databases may be stored on an external server for retrieval by the Data Access Layer 130 via the API server 120.

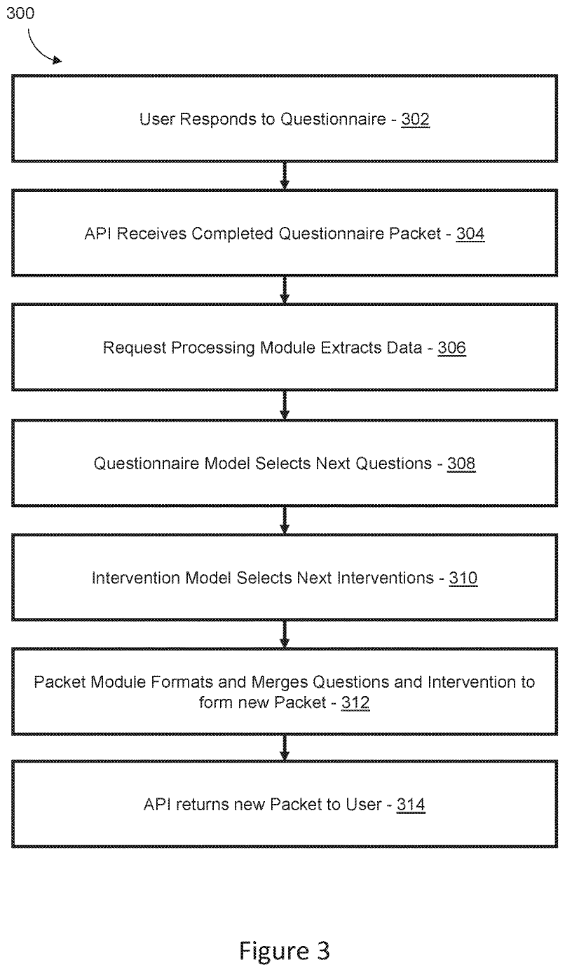

QID 134 is implemented as a YAML-based question bank for “dynamic_health”, comprising 23 major topics, 90 subtopics and 500 potential questions. It should be understood that the term ‘question’ described herein refers to a data object which may include, in addition to the question per-se, question metadata associated with the question. The question metadata may comprise, for example, one or more of: a question-type indicator; weighting fields; positioning/eligibility fields; and a runtime topic-weight modifier. As described later, for example, with reference to FIG. 3, the question metadata allows for improved filtering and weighting of questions throughout the system 100. A sister directory includes snapshot data for “dynamic_advice” or “dynamic_goals” comprising 175 possible interventions. Preferably, these are loaded into in-memory data structures, such as Pandas DataFrames, each time the application starts.

Questions or interventions are categorised within the QID 134 to belong to at least one category relating to physical or mental health. These health categories may be divided into more specific health topics, and these categories and topics may be associated with risk scores comprising an end users risk profile. Topics relating to physical health may comprise: diet; physical activity; hydration; mindful eating; caffeine intake; sugar intake; nicotine use; alcohol consumption; pain conditions or musculoskeletal conditions. Similarly, further topics relating to mental health may comprise: sleep; social awareness; attention and focus; behavioural impact; social conduct; social norms; addressing unresolved emotional issues; cultivating resilience; building self-awareness and introspection; mental health care; stress management; depression; social connections; or mobile phone and social media use. New topics and categories may be added or withdrawn depending on additional factors, for example, an end user or a 3rd-party client 160 choosing to remove “alcohol-related” questions. Conversely, a separate end-user or a 3rd-party client may request a topic comprising “alcohol-related” questions to be added for their company in category related to occupational health.

Interventions may range from short-form advice (“Take a 5-minute walking break”) to multi-day goals (“Achieve at least 8,000 steps daily for the next 4 days”). Preferably, the system 100 scores each intervention's potential to help a specific user by which item is most likely to resonate with an end user. Over time, if end user follow-ups indicate that certain advice is not effective, the model lowers the priority of that intervention.

The system also comprises a Processing Engine 140, which is responsible for computing new sets of questions and interventions for an end user, as well as updating at least one risk profile associated with an end user. A risk profile may comprise at least one risk score associated with an end user. Risk scores may be a single score associated with a user. Alternatively, multiple risk scores may be assigned to a user, where scores may be associated with a given health category (i.e. physical or mental health), or they may be associated with a given health topic (i.e. nicotine use, depression).

Risk scores comprising a risk profile associated with a user may be calculated partly based on external information comprising clinical data, such as data comprising mortality, comorbidities and other health outcomes, from longitudinal studies. Risk scores may also be calculated based on health metrics provided by an end user, a 3rd-party client 160 or otherwise determined by the system based on responses to questions or interventions. Health metric data may comprise one or more of: age; gender; weight; height; geolocation data; intraday steps; daily step cadence; sleep patterns; heart rate; VO₂; METs; physical activity, rest and recovery patterns; tobacco and/or alcohol consumption; dietary habits; social and behavioural health; mental wellbeing or mood monitoring. Risk scores may be determined from a suitable machine learning algorithm. Said machine learning algorithm, or indeed others used elsewhere in the system, may be trained on data provided by a 3rd-party client 160, external information comprising clinical data or the health metrics by an end user. The machine learning algorithm may also be trained with end user responses to provided questions or interventions or other population-level metrics determined by the system 100.

Data Access Layer 130 provides stored data to the Processing Engine 140 for use in determining questions or interventions and/or updating risk profiles. Such data for use may comprise biometric data, personal data, health risk profiles or insurance data, as described above. Data for determining questions or interventions comprises questions or interventions completed by an end user, or questions or interventions provided to an end user by the system 100. Alternatively, the Processing Engine 140 may receive data for determining questions or interventions and/or updating risk profiles directly from the API server 120, which may in turn request said data from the client device 150.

Processing Engine 140 also receives potential questions or interventions from the QID 134. Once questions or interventions have been determined and/or risk profiles have been updated, the Processing Engine 140 then provides newly determined questions or interventions and/or updated risk profiles to the Data Access Layer 130, for storage in the UCJD 136. Alternatively, the Processing Engine 140 may instead provide determined questions or interventions directly to the API server 120 for transmittance to the client device 150.

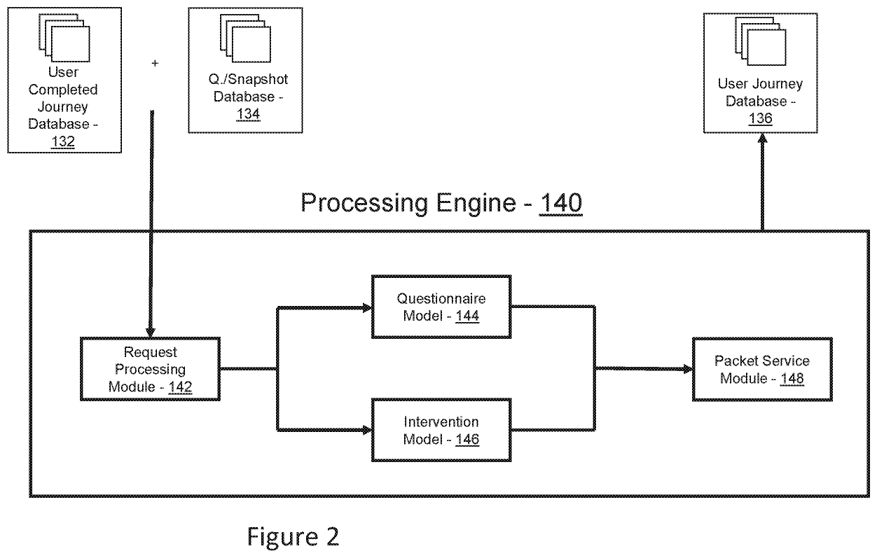

FIG. 2 shows a schematic diagram of the Processing Engine 140, along with subcomponents of the Processing Engine 140, and the relationship between the Processing Engine 140 and the databases comprised in the Data Access Layer 130.

The subcomponents of the Processing Engine 140 comprise a Request Processing Module 142, a Packet Service Module 148, and at least one model for selecting questions and interventions for provision to a user. In said embodiment, the at least one model for selecting questions and interventions further comprises a Questionnaire Model 144 and an Intervention Model 146.

Request Processing Module 142 is responsible for processing data received or requested from the Data Access Layer 130. Said data may relate to incoming end user responses to questions or interventions, timestamps or progression through question steps or stages. Processing said data may involve further transforming or normalising the data into a standardised format for producing questions or interventions. Alternatively, the Request Processing Module 142 may directly receive or request data via the API server 120.

As will be appreciated, Request Processing Module 142 may instead comprise, supplement or replace functionality covered by the Data Access Layer 130, in relation to managing incoming data to the Processing Engine 140, such as loading or merging data to or from any database in the Data Access Layer 130.

Questionnaire Model 144 and an Intervention Model 146 control the selection of new questions and interventions, respectively. The questions may be constructed to interrogate factors associated with known health conditions using population statistical data and/or medical datasets (e.g. from a national biobank, health service or national statistical survey). The questions may then be associated with these health conditions in said database(s). The selection of questions may be at least partially based on a history of questionnaires or interventions previously completed or attempted by a user. Preferentially, relevant questions or interventions are selected from said database(s) using an appropriate machine learning algorithm, by the Questionnaire Model 144 and the Intervention Suggestion Model 146. For instance, the machine learning algorithm may comprise a regression model with pre-determined or learned weights for a question set or health topic that may relate to, for example, the risk of developing a given category of health conditions or an overall risk score associated with an end user.

Weights comprise the risk profile with individual risk scores determined for a given question set or health topic. The same risk profile for an end user may be used to select both questions or interventions. Alternatively, a separate risk profile may be used to select questions, and another may be used to determine interventions. Weights for a question or an intervention may be adjusted based on the previous responses within an overarching topic for a health category. Weights may also be manually configured by the end user or determined from or otherwise adjusted using data provided by a 3rd-party client 160. Such data provided by a 3rd-party client 160 may include physical activity information derived from apps associated with wearable devices, such as heart rate (HR), the number of steps taken a day, maximal oxygen consumption or aerobic capacity (VO2), or metabolic equivalent of task (MET). Other forms of data for determining these weights are anticipated, such as geolocation data. As will be appreciated by the skilled person, other suitable machine learning algorithms may be contemplated in this context and in others, such as a neural network(s) or a decision tree(s).

Questionnaire Model 144 calculates a set of candidate questions to deliver to a user by referencing a weighted probability function which adjusts the odds of each next question being presented based on the questions the user has already answered. In some examples, prior to determining the set of candidate questions by referencing the weighted probability function, eligibility filtering is performed to filter candidate questions. The eligibility filtering is described in more detail with reference to FIG. 3. The weighted probability function comprises an end user's prior responses and a contextual risk score (R). The contextual risk score comprises an overall risk score for an end user, or a risk score for a given health topic.

Let Q be the set of available questions, each question qᵢ in Q having a topic weight (w₍tᵢ₎), a base question weight (w₍qᵢ₎), and a user-configured topic multiplier (w₍tcᵢ₎). Each response history entry may additionally contribute an answer-based adjustment term (1−w₍aᵢ₎), where w₍aᵢ₎ is the recorded answer weight for question qᵢ. The response history adjustment factor may be computed by aggregating answer weights over a configurable historical scope, where a scope size and an aggregation method can be loaded from a database, configuration channel or research-derived setting rather than being hard-coded. For example, answer-based adjustment term (1−w₍aᵢ₎) may be replaced by a topic-aggregated term ρ_{τ(i)}(t). Where the topic-aggregated term for a candidate question qᵢ of topic τ at iteration t, ρ_{τ}(t) is equal to

1/|Last_{N}(H_{τ}(t))|×Σᵣ_{_{∈}}_{lastN(Hτ(t))}max(1−a_{w}(r),λ),

where N refers to a historical scope (also referred to as a sliding window size), last_{N}(S) takes a most recent N elements of ordered set S (or where there are not N available, all available elements), H(t) is a user's response history up to iteration t, H_{τ}(t) refers to the subset of H(t) whose questions belong to topic τ, λ is the response weight threshold and a_{w}(r)∈_{[}0, 1] represents the answer weight recorded for response r∈H(t).

Beneficially, this allows tuning of how much previous answer history matters when deciding what to ask next. The Questionnaire Model 144 then assigns each question qᵢ a selection probability Pᵢ obtained by normalizing the product of these factors in proportion to the user's current risk score R. R may calculated from a combination of biometric data, personal data, health risk profiles or insurance data, as described above.

In one example, a simplified equation for Pᵢ is shown in Equation (1):

Pᵢ=((1−w₍aᵢ₎)×w₍qᵢ₎×w₍tᵢ₎×w₍tcᵢ₎×R)/Σⱼ((1−w₍aⱼ₎)×w₍qⱼ₎×w₍tⱼ₎×w₍tcⱼ₎×R).

Of course, if ρ_{τ}(t) is used in replacement of the answer-based adjustment term (1−w₍aᵢ₎), the skilled person will understand that equation 1 becomes a selection probability Pᵢ(t) for candidate qᵢ of (ρ_{τ(i)}(t)×wₜ(qᵢⱼ)×wₜ(tᵢⱼ)×wₜ(tcᵢⱼ)×R)/Σⱼρ_{τ(j)}(t)×wₜ(qᵢⱼ)×wₜ(tᵢⱼ)×wₜ(tcᵢⱼ)×R) since that the topic-aggregated response-history factor multiplies the per-question base weight, the topic weight, the user-configured topic multiplier, and the contextual risk score, with normalisation across all eligible candidates.

Intervention Model 146 determines whether any interventions should be chosen from the available set of interventions. These interventions are similarly treated as conditional elements within the set Q (i.e., Q includes both standard and intervention items) but are only enabled when preceding responses meet certain criteria—such as w₍aᵢ₎ exceeding a threshold or the user's risk score R surpassing a specified limit.

Specifically, let S represent the set of all interventions that are conditionally available based on earlier responses. Each snapshot sᵢ∈S is associated with a base intervention weight w₍sᵢ₎, and an eligibility indicator Iᵢ∈{0, 1}, which equals 1 if the intervention's prerequisite conditions are satisfied, for example, if an end user's risk score R surpasses a threshold or prior answers match a designated pattern. The Intervention Model 146 then assigns each snapshot sᵢ a selection probability Sᵢ computed via an equation analogous to Pᵢ, shown for example in Equation (2) below:

Sᵢ=((1−w₍aᵢ₎)×w₍sᵢ₎×Iᵢ×R)/Σⱼ((1−w₍aⱼ₎)×w₍sⱼ₎×Iⱼ×R),

where the denominator sums over all interventions sⱼ∈S to ensure normalization. Once an intervention sᵢ is delivered, it may in turn prompt an additional follow-up if, for example, its measured impact or user engagement exceeds a predetermined threshold, thereby ensuring that high-priority interventions remain active and that the user continues receiving pertinent advice or goals until measurable improvement is observed.

Questions or interventions have associations with each other, wherein the selection of a given question biases the selection of associated questions or interventions. Preferably, said associations are grouped by a given health topic or domain, such as cardiovascular disease. The strength of such associations between questions or interventions is parameterised, wherein said parameters are determined through causal analysis of questions that are frequently asked in an ensemble to a particular user, or through analysing the questions answered by networks of users with similar health conditions. In some instances, the associations between questions may not be grouped by a given health topic or domain but by a learned response. For example, upon provision of a plurality of questions and answers form a plurality of users to a machine learning model for training, the machine learning model may learn that individuals that give a particular answer to a first question are probable to give a particular answer to a second seemingly unrelated question. Such associations are also captured.

Questionnaire Model 144 and the Intervention Model 146 incorporate couplings Tᵢⱼ at the topic level (and at an inter-subtopic level), representing pairwise or multi-way interdependencies among topics. In particular, the final adjusted weight for question i∈Q is multiplied by a factor Exp(tᵢⱼ) for every relevant topic j, where Tᵢⱼ is a learned parameter derived from historical patterns and outcomes. These parameters are determined through a causal analysis or network estimation of user responses, capturing the directed and conditional dependencies among topics and quantifying how manipulating one topic's weight will causally influence responses to others. Alternatively, these parameters may be determined through a machine learning algorithm trained on previous responses to questions and interventions as well as risk scores for health topics, as determined from a population of end users. Consequently, questions addressing correlated health domains can be dynamically scaled upward or downward, providing a more contextually accurate questionnaire flow that accounts for real-world coupling effects among various health and lifestyle factors.

Couplings may also be incorporated using nested Boolean eligibility wherein certain questions or interventions are only accessible if a combination of required answers are present. A data structure in such nested Boolean eligibility may be: ‘list[list[answerKey]]’ having semantics:
Outer list=AND: every group must pass
Inner list=OR: at least one answer key in that group must be present
Empty marker [[ ]]=no condition, therefore trivially true

For example, an intervention to “create realistic alcohol reduction goals” may only be eligible if a user has given a correct specific answer to “Recognising problematic alcohol behaviour”, and provided one of a correct answer of a plurality of possible answers to a question “Provide a benefit of moderate drinking”.

Intervention Model 146 comprises a machine learning algorithm that learns groups of interventions that a user has previously completed successfully, or learn that a user responds positively to certain sets of interventions. Preferably, the system may then provide these sets or groups of interventions to an end user. Conversely, the Intervention Model 146 deprioritises sets or groups of interventions that are found by said machine learning algorithm to be completed less often by an end user.

Processing Engine 140 is additionally configured to provide a “pathway”, comprising a group or set of questions or interventions relating to a given health outcome or topic to be completed by an end user. For example, a pathway relating to improving mental health outcomes may comprise a group or set of questions or interventions relating to mindfulness practice and/or reducing nicotine or alcohol intake. Said pathway is determined based on user responses to questions or interventions, their overall risk score or risk score for a given score for a given category of health outcomes. A plurality of different pathways may be associated with a given user with the pathway currently being interrogated referred to as an active pathway.

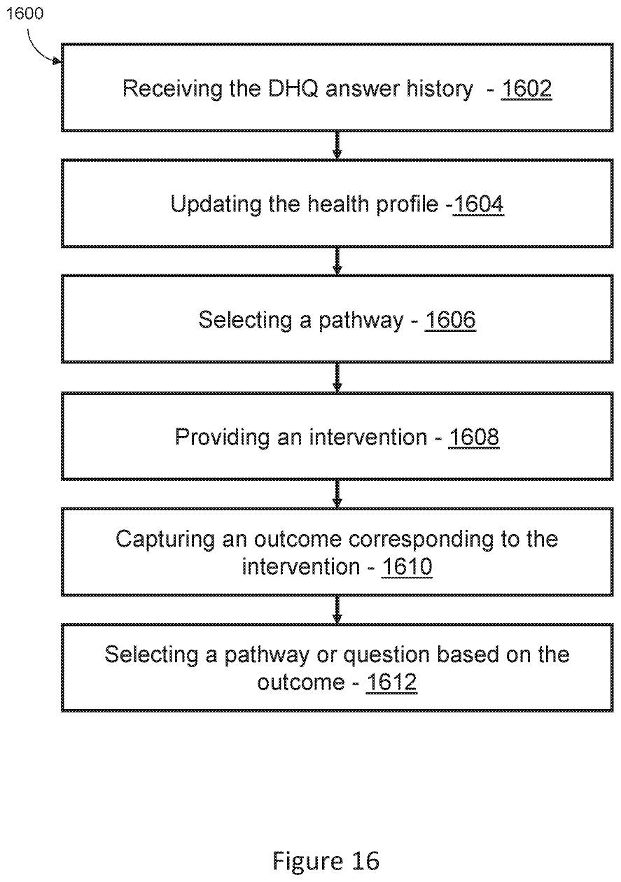

In some examples, the questionnaire model 144 feeds into a pathway model such that responses from the questions and corresponding change to user profile inform a pathway sequence, as shown for example, by method 1600 in FIG. 16. At step 1602 the question answer history is received and at step 1604 the user health profile is updated based on the question answer history. At step 1606, a pathway is selected. In some examples, the pathway model selects a user pathway as an active pathway based on the current health profile and response history, in order to deliver an intervention via the intervention model 146. The pathway model then uses the user's subsequent behaviour and health profile updates from question answers to determine a next pathway step. In this way, the pathway is not static, but instead evolves with responses to questions. Furthermore, the pathway may change in response to a user interacting with an intervention, for example, in response to a user accepting, ignoring, completing or failing an intervention.

At step 1608, the intervention model 146 provides an intervention to the user. In some examples, the pathway model moves a user through a structured sequence of interventions where interventions may range from short-form advice (“Take a 5-minute walking break”) to multi-day goals (“Achieve at least 8,000 steps daily for the next 4 days”). For example, a set of interventions corresponding to a pathway may be an advice intervention advice, then a behavioural goal, then a goal follow-up. where each intervention has a different purpose on the pathway. The pathway model may alter the pathway based on goal return, completion and user health response. For example, after a goal is delivered, the next pathway decision can depend on whether the user returned to the goal, whether they completed it over a defined period, and whether later health answers moved in an intended or predicted direction. In this way, system 100 does not only record an outcome; it uses that outcome to select the next intervention or follow-up.

An intervention impact may be determined through a feedback loop between the pathway model and through subsequent question answers. The system 100 can use later question answers to assess whether an intervention appears to have changed the relevant health outcome. For example, a sleep-related goal can be followed by related questions (such as sleep or energy related questions), and the answers can influence whether the system 100 repeats, escalates, changes, or stops that pathway. Burden-aware pathway selection can be implemented using eligibility filtering such that the pathway model takes account of user burden, fatigue, recent activity, or likelihood of completion when selecting the intervention. The system 100 does not just determine what would be theoretically best but determines whether it is appropriate to ask of that user at that point in the journey. For example, whilst the most theoretically best intervention may be advice to reduce alcohol consumption, the system 100 may recognise that a user is not currently susceptible to severe advice and instead present, as a next pathway step, a bedtime goal for the week.

Escalation, de-escalation and substitution of interventions, such as goals may be executed by the pathway model to escalate to a more demanding or more targeted goal or intervention. Conversely, if a user does not complete a goal, the system may de-escalate, provide easier advice, switch topics, or select a substitute goal. The pathway model may also inform the pathway based on aggregated responses and completion data from similar users or cohorts. That means the system 100 can learn which goals or interventions tend to work for particular user states, rather than relying only on a static ruleset. Beneficially, the use of population-level intervention outcomes to adjust future behavioural pathway choices for an individual user provides a more efficient pathway optimisation with improved completion rates and fewer steps to a particular beneficial outcome.

The pathway model may change pathway content, sequencing rules, thresholds and topic weightings through configuration rather than hard-coded application logic. That allows the system 100 to rebalance topics, introduce new goals, change follow-up timing, or adjust pathway rules without rebuilding the underlying engine. This provides a system that is a configurable adaptive engine rather than a fixed questionnaire or fixed recommendation tree.

At step 1610, an outcome corresponding to an intervention is captured. A user's interaction with an intervention may influence not only the next intervention, but also the future question flow. For example, failure to complete a goal may cause the system to ask more diagnostic questions around blockers, while successful completion may reduce the need for repeated questions on that topic. This is useful because it provides a closed the loop both directions: question answers drive pathways, and pathway outcomes drive question selection. Different goals or interventions can have different follow-up windows. Some behaviours may be checked quickly; others only make sense after a longer interval. The system can therefore select not only what to follow up on, but when the follow-up should happen. Subsequent pathways or questions are selected based on the captured outcome in step 1612 and the method 1600 iterates through steps 1602-1612. Processing Module 140 comprises a Packet Service Module 148, which is responsible for formatting received questions and interventions into a suitable format, such as JSON or YAML. The questions and interventions may be additionally packaged with further data, such as timestamps or checksums, which may then be further sub-divided into a series of packets in preparation for transmittance to a client device 150 or a 3rd-party client 160. The Packet Service Module 148 may then provide the merged questions and interventions directly to the API layer 120 or as a series of packets for transferal.

In some embodiments, the Processing Engine 140 comprises a machine learning algorithm that is trained on historical user responses to determine correlations and causal links between health outcomes and responses to questions or interventions. Such correlations and/or relationships comprise: which questions or interventions are linked directly or indirectly to certain subsets or clusters of user groups; which questions or interventions are successful in changing user behaviours over time; or the timeframes required to enact changes in lifestyle or health behaviours. For example, the machine learning algorithm may determine that individuals who report moderate stress and a high daily step count benefit from mindfulness practice, and consequently, the Processing Engine 140 increasingly selects questions or interventions that relate to mindfulness for similar end users.

The Processing Engine 140 also selects specialised questions or interventions relating to a 3^{rd}-party client 160 if certain conditions are met, or the system may signpost an end user to a given 3rd-party client, if an end user's health outcomes receive a poor forecast.

In some embodiments, the Processing Engine 140 is configured to provide reports covering physical and mental health trends for given categories of an individual, such as age, gender or workplace. The Processing Engine 140 is further configured to identify anomalous individuals or groups of individuals based at least on differences from an optimal health status. The Processing Engine 140 may be configured to link these reports to health outcomes for individuals, or the system may communicate with a 3rd-party client 160 to verify that a specific intervention associated with a 3rd-party client 160 has been completed.

The Packet Service Module 148 may also provide the packaged data to the Data Access Layer 130, for incorporation into any database associated with the Data Access Layer 130, such as a database for provided questions or interventions sent to a user, for example, the UJD 136. It will be appreciated that, when data relating to personal health is transferred to the 3rd-party client 160, data may be provided at an appropriate level of granularity, which may be set by the user, to ensure privacy of personal information.

Packet Service Module 148 may be implemented as a suite of utility classes that merge or reformat question/answer data into consistent “packets” for GraphQL responses. Packet Service Module 148 may additionally incorporate additional modules that handle string manipulation or key parsing, ensuring consistent data references.

In some embodiments, the Packet Service Module 148 is further configured to transmit new questions and interventions to an end user according to an interval setting. This interval setting may be stored on or determined by the client device 150, the user session 110, or by the Processing Engine 140, and can be accessed when the Processing Engine 140 determines a new set of questions and interventions. This interval setting is pre-configured based on user input, using the client device 150 or the user session 110. Alternatively, this interval setting may be determined dynamically through a separate algorithm, such as a suitable machine learning algorithm, based at least partially on historical questions or interventions previously answered by an end user.

Processing Engine 140 is additionally configured to track the effect of seasonality on given risk factors or health outcomes, and to tailor risk profiles based on seasonality information. For example, the Processing Engine 140 may track responses to mental health questions in winter months and adjust the weighting of risk profiles using the Request Processing Module 142 accordingly. Questions or interventions are, as a consequence of the adjusted weighting, also be selected based on seasonality information. For example, questions relating to mental health may be preferentially provided to users in the winter months, where low light conditions have an impact on mental health. As another example, interventions relating to step count may be preferentially selected in summer months, where temperate weather increases the likelihood that a user chooses to attempt that intervention.

More generally, the intervention model may:
Use user health profiles to generate personalised intervention strategies, including advice, goal-setting, and benefits recommendations
Perform adaptive learning of interventions, ensuring delivery at the right time and in a personalised manner to optimise health impact
Provide intervention pathways that make recommendations from user health profiles to available third-party services (e.g., EAP, benefit platforms, company-specific perks), delivered in a similarly personalised fashion
Generate interventions incorporating modified in-app game mechanics, such as customised quests and challenges (e.g., walking or meditation), with dynamically adjusted incentives and recommendations based on the user's profile.

In some embodiments, the Processing Engine 140 generates further predictions related to health outcomes, before sending them to a client device 150 or a 3rd-party client 160 using the Packet Service Module 148 and via the API server 120. These further predictions may relate to the risk of developing a given health condition, such as: cardiovascular conditions; diabetes-related complications; pneumonia and respiratory disease; cancer diagnoses; mental health disorders; musculoskeletal conditions; sleep disorders or depression; anxiety; or burnout. Predictions are used by the Processing Engine 140 to derive additional scores or metrics, which may comprise: present and future claim costs for health insurance policies; biological age of an individual; or rankings of groups of users based on their health or risk profiles. Based on these further predictions, the system 100 may be further configured to provide an alert or further recommendations to a client device 150 associated with an end user in the event they have a predicted adverse health outcome.

The system 100 may be implemented according to a series of methods, for example, files relating to specific functions comprising methods for performing the function written in the Python programming language that enable functionality of the constituent components and sub-components. For example, a Python file ‘run_survey_journey_graphQL.py’ may launch GraphQL endpoints and orchestrate user queries. As a further example, a Python file ‘health_questionnaire_model.py’ may comprise core logic for question selection. As a yet further example, a Python file ‘health_intervention_model.py’ may comprise core logic for interventions. As a yet further example, a Python file ‘health_questionnaire_model_tools.py’ comprises helper services for merging question and intervention sets as well as formatting results. As a yet further example, a Python file ‘request_processing.py’ may transform user inputs into workable data structures, such as Pandas DataFrames. As a yet further example, a Python file ‘key_utils.py’ may comprise functionality relating to string key generation and parsing.

FIG. 3 demonstrates an example user interaction and data flow through the system 300 for the selection of new questions and interventions to be provided to an end user. Preferably, questions are provided to a user via a user interface on the client device 150. Similarly, user responses are received by the user interface.

In one example, a user responds to questions provided to them via a user interface of the client device 150, as well as noting which interventions they have attempted 300, both of which are sent from the system 100 to the client device 150 via the user session 110, which is accessed using the end user's client device 150. The user responds to the questions provided to them by providing an input the user interface of the client-device 150. The input to the user interface may be, for example, a selection of one of a plurality of multiple choice options, a text input or an audio response.

Upon completion of the questions or interventions, the client device 150 logs the responses received by the user interface and sends a request to the API server 120 comprising a packet 302, which in turn comprises responses to questions and the interventions, as well as a user ID, for identifying the user session 110. The API server may then provide this packet directly to the Request Processing Module 142 of the Processing Engine 140. The Request Processing Module 142 then extracts information related to completed questions and interventions 306, as well as the health risk profile associated with an end user, which may then be used to update weights in both the Question Model 144 and Intervention Model 146. Alternatively, the Data Access Layer 130 first stores incoming information related to the completed questions or interventions in an appropriate database, such as the UCJD 132, before providing it to the Request Processing Module 142.

Consequently, the Question Model 144 selects new questions from the set of available questions 308, which is provided from the QID 134 of the Data Access Layer 130. Similarly, the Intervention Model 146 then selects new interventions from the set of available interventions 310, which is also provided from the QID 134. Alternatively, the Question Model 144 may select questions 308 in parallel with the selection of interventions 310 by the Intervention Model 146. As a further alternative, interventions may be chosen before questions, or one or the other may be omitted from selection.

In some embodiments, questions or interventions may be selected 308-310 either partly or fully randomly, or deterministically based at least partly on previous questions that have been presented to a user. Deterministic selection has an emphasis on showing an end user questions or interventions from health topics that are less frequently asked or with a higher risk score for that user. Questions or interventions may also be chosen based on a specific facet of user health, such as the likelihood of relapsing into behaviours associated with poor health outcomes, determining the longevity or persistence of certain behaviours, or determining a user's receptivity to a given intervention. For instance, should a given intervention associated with a particularly high weight or risk score be selected, it may cause additional follow-up questions to be selected, relating to whether a user has attempted or completed said intervention.

The questions and corresponding responses may have associated temporal data. For example, a response may comprise an answer to the question and a date time stamp of the answer. The temporal data associated with the questions and answers allows a temporal user profile to be constructed. It also enables a likelihood of relapse to be determined based on the temporal data by forecasting the historical temporal data obtained from the questions and corresponding responses into the future.

In some embodiments, questions or interventions are further selected or provided based on set time intervals or in groups. This is referred to as eligibility filtering with reference to FIG. 1. The eligibility filtering may filter a candidate question pool question by question prior to provision of the filtered questions to the weighted probabilistic function. For example, questions relating to smoking or alcohol consumption may provide better insight to a user when asked regularly whereas questions related to more chronic or seasonal health issues may need to be asked less regularly. Furthermore, the change is user answers over time may be a greater or lesser indicator of user susceptibility to intervention or likelihood of relapse for certain questions and not others. These time intervals or combinations are learned by an additional machine learning algorithm that is implemented in the processing engine or elsewhere in the system, with a view to maximising the number of successfully completed interventions by a user. For instance, said machine learning algorithm, after being provided data related to completed or failed interventions and the timing of their provision, may learn intervals that space out interventions involving reaching a certain step count, based on a user's past record of completing interventions related to step counts.

In some embodiments, it may be determined that a user is more susceptible to a certain question type or presentation of question. The susceptibility to question types may then be used by the system to determine which question types to present to the user. Furthermore, the system may reward users for responses to questions and determine user susceptibility to rewards. For example, a first user may become twice as likely to respond to a question type they are not typically susceptible to answering if provided with a reward whereas a second user may be agnostic to reward but only susceptible to certain question types, such as multiple choice rather than free text questions. In such an example, the system may provide more rewards to the more first user. Rewards may be, for example, vouchers, in-app coins which may be exchanged for money or access to certain sporting or wellness benefits.

In some embodiments, the Processing Engine 140 determines a typical completion time for each question or intervention that it may provide to a user. This typical completion time is determined using an aggregate of many user values related to timestamps of completed questions or interventions. The typical completion time may then be used by the system to determine whether the user is progressing quickly or slowly through a set or group of questions or interventions. For example, if the system determines that the user is likely to respond slowly or is less engaged, it will reduce the number of questions or interventions offered. Conversely, an end user with a history of fast response times may receive a larger set of questions or interventions if they demonstrate an ability to handle more queries without disengaging. In an alternate embodiment, the system may utilise a suitable machine learning algorithm to determine the typical completion time. In some examples, the eligibility filtering may filter questions based on the completion time for the question. In some examples, questions are filtered based on a cooldown criteria which assign an interval based on question type or time taken to answer the question. In such an example, if the interval has not elapsed, the question is filtered out before probability weighting, in this way, the system avoids asking repetitive or burdensome questions too often. For example, particularly long questions may have a cooldown criteria of 20 minutes so only one long question is asked in a 20 minute session and the subsequent long questions may be filtered out in the eligibility filtering step.

Once questions and/or interventions have been selected by their respective model, then the selected questions and/or interventions are changed into a suitable format, such as JSON or YAML, by the Packet Service Module 148. Once formatted, the questions and/or interventions are merged into a packet alongside additional data, such as checksums and timestamps, for transferal 312 to the client device 150. Once a new packet has been creating comprising newly selected questions and interventions, the API server 120 then transfers the packet to the client device 150, for future completion by the end user.

Data flow through the system 300 may additionally incorporate additional filtration of new questions and interventions before provision to an end user. The Questionnaire Model 144 and the Intervention Model 146 may be further configured to filter questions or interventions that have been provided to an end user too recently, or those that have been disqualified by prior answers. For example, questions or interventions relating to nicotine use will be disqualified if an end user does not smoke nicotine. Selection of questions or interventions as outlined above contributes to a reduction in repetition for those questions, as well as sampling more questions related to higher risk health topics for categories for an end user.

Data flow through the system 300 may additionally incorporate an updating of a risk score or risk profile associated with an end user. In at least one embodiment, the system recalculates an end user's associated risk profile in substantially real-time using the Processing Module 140. Recalculation of an end users risk profile may occur at set intervals, or may be performed upon registering particular responses to questions or interventions. For example, if an end user drastically reduces their step count, the system may determine heightened risk profiles for relevant conditions, health topics or health outcomes. As a consequence, questions or interventions related to those health topics or outcomes have a higher probability of being selected in future instances of question or intervention selection. Recalculation of risk profiles may be an iterative process, wherein multiple rounds of questions and interventions are fine-tuned over time to particular health topics or outcomes of greater relevance to an end user.

In some embodiments, data flow through the system 300 additionally incorporates detection of when an end-user provides comparatively low or lower quality, incomplete or otherwise untrustworthy data. Detection of low-quality data occurs based on determining abnormal patterns of responses, such as when an end-user responds to questions in too short a time, or chooses the first answer repeatedly in a questionnaire. Such detection occurs as and when a user session 110 is open, or as post-hoc analysis over previously completed questionnaires and interventions stored by the Data Access Layer 130. The Processing Engine 140 then re-weights any associated questions or calculated risk profiles. Alternatively, the Processing Engine 140 may interpolate data in the event that a pattern of missing responses is detected, such as when a user does not answer a group of questions relating to a given health behaviour.

In some embodiments, the system 100 continuously updates the user's risk profile during data flow through said system 300. The system is configured to update risk profiles biometrics and other data related to the health of an end user, such as steps, accelerometer data, heart rate or and insurance updates. For instance, if a user has drastically decreased their daily step count, the system recognizes heightened risk for certain conditions, adjusting question selection to explore that area or propose relevant goals.

Data flow through the system 300 may additionally incorporate provision of incentives to encourage completion of provided questions and interventions. Incentives may be in the form of a suitable in-app currency, or may be associated with services provided by a 3rd-party client 160 linked to the system. The provision of incentives is controlled by the user session 110. The system then further determines or tracks which incentives are used by an end user. To further drive user engagement, the system 100 comprises an additional component in the user session 110 supporting chatbot functionality within a user session, to provide advice, goals, or answer questions related to health behaviours. Said chatbot may also act as a wrapper for questions or interventions that are posed to a user. Such a chatbot may be implemented, for example, using a large language model (LLM).

In some embodiments, to further drive user engagement, interventions incorporate elements from in-app gamified mechanics, provided on the client device 150. For instance, an app may provide personalised challenges to complete a given intervention or set of interventions. Successfully completing said challenge may provide an additional incentive, such as those noted above, wherein the total amount of a given incentive may be altered dynamically based on a user's profile.

In some embodiments, end users are provided graphical summaries of their performance or responses to provide an overview of their mood and/or wellbeing, as determined by the Processing Engine 140 and provided on the client device 150. Users may also see personalised recommendations or advice outside the scope of particular interventions, based on their responses to questions or interventions.

Data flow through the system 300 may additionally determine the engagement of an end user with provided questions and interventions. A corresponding engagement metric is determined as the user responds to questions or interventions. The engagement metric may be compared to a threshold, which determines when questions or interventions are no longer provided. For example, the engagement metric comprises a measured time to respond to a given set of questions or interventions.

Data flow through the system 300 may additionally:
cause targeted questions to be asked after a user attempts an intervention;
incorporate accessing of external data associated with a 3rd-party client 160; and
incorporate merging external data associated with a 3rd-party client 160.

In some alternatives, data flow through the system 300 may additionally incorporate selecting specific interventions associated with a 3rd-party client 160. For example, the system 100 may enable specialized questions, if an end user's policy with a 3rd-party client 160 that provides insurance includes certain health programs. As a yet further example, the system 100 may provide relevant signposting to Employee Assistance Programs, such as mental health programs, before health problems become severe. Where a 3rd-party client 160 is an insurance provider, the system 100 may also validate coverage for specific interventions. In at least one embodiment, the system may be further configured to provide recommendations to 3rd-party clients based on either a user's health profile, risk profile, or based on the interventions that a user is attempting or has completed. The system 100 may communicate to 3rd-party clients to ensure that any associated services that are provided complement the provision of interventions or incentives to an end-user. As an example, the system 100 may provide a series of interventions relating to mindfulness to an end-user, before directing them to an associated 3rd-party client 160 that supports meditation or mindfulness exercises.

In some alternatives, data flow through the system 300 may additionally incorporate providing data to a 3rd-party client 160 based on completed questions and interventions.

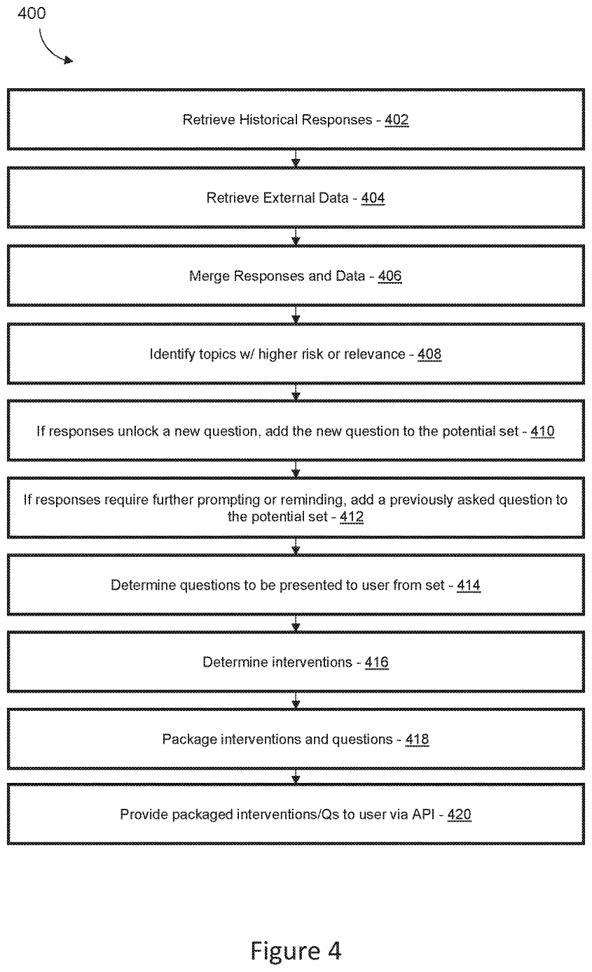

FIG. 4-7 show additional variations of data flow through a system 300, which comprise additional steps to the main data flow of determining questions or interventions based on user response, which may be performed by the components of the system 100 disclosed above.

FIG. 4 demonstrates an example data flow 400 with additional filtering and prioritisation of questions based on identification of topics with higher risk or relevance, specifically, 406-410.

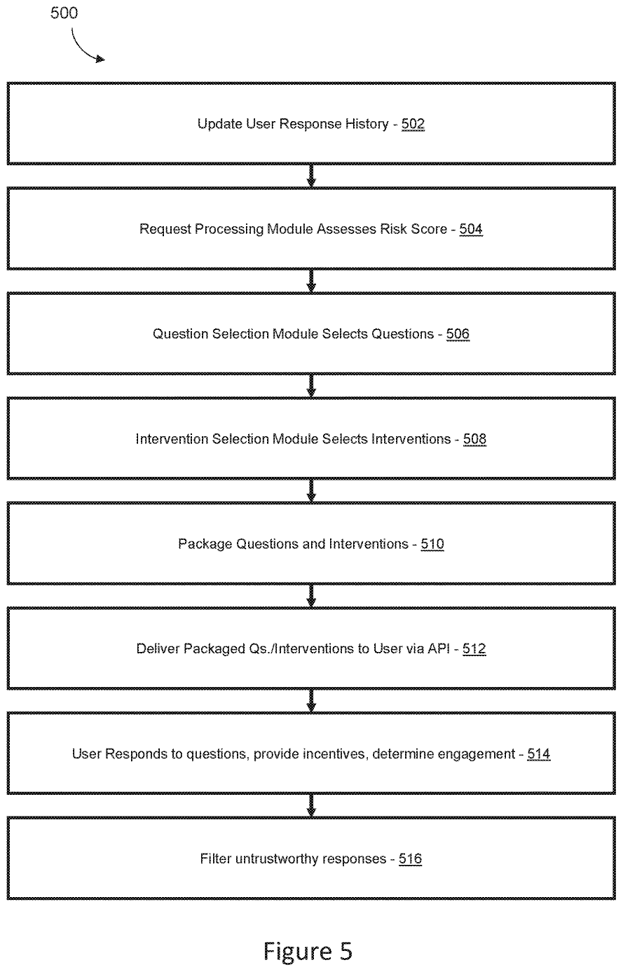

FIG. 5 shows an example data flow through a health system 500 with additional determination of user engagement 512, and provision of incentives 514, after updating risk scores based on user responses 502.

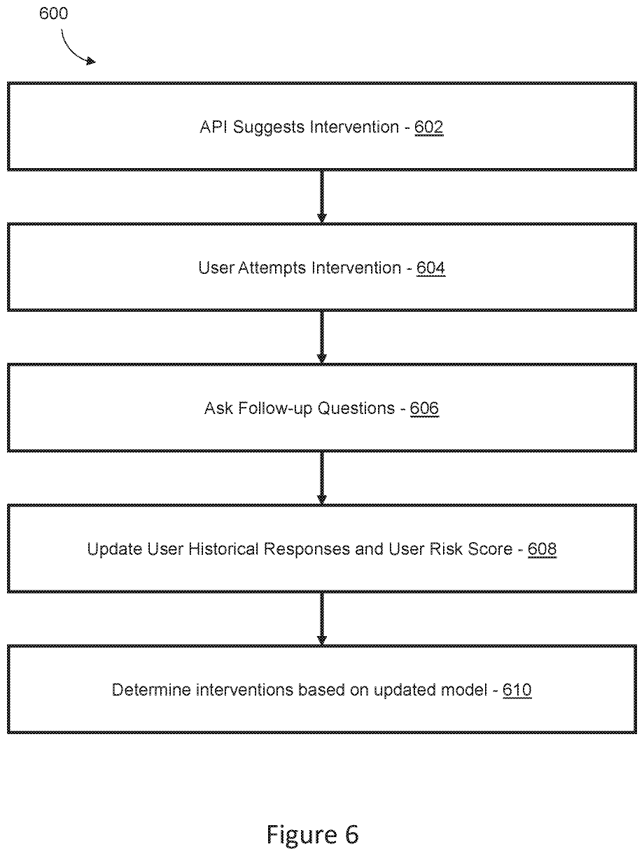

FIG. 6 shows an example data flow through the system 600, where interventions are suggested 602, the user attempts them 604, follow-up questions are asked to a user 606, before updating the user risk score 608, and re-determined interventions based on the updated user risk score 610.

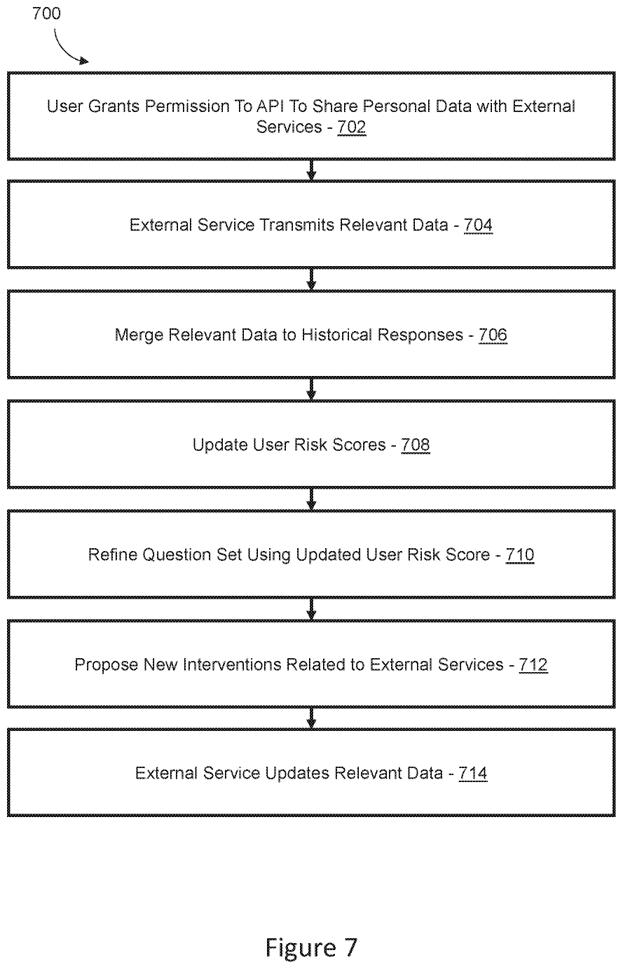

FIG. 7 shows an example data flow 700, wherein an end user grants permission for data sharing 702, external data related to a 3rd-party client 160 is provided 704 to the system 100, which is merged into historical data comprising previous responses 706. The system then updates user risk scores 708, before proposing new interventions 712 relating to the 3rd-party client 160. The 3rd-party client 160 may then update the system with any relevant data determined by said 3rd-party client 160.

As will be understood from these illustrative examples, modifications from each may be included within the context of data flow through the system, as demonstrated by FIG. 3, wherein additional steps are included, or specific steps are excluded. Furthermore, it will be appreciated that feedback can occur from one stage of the data flow to a prior stage. For example, where answering questions or interventions by an end user leads to updating of the risk profile before selection of new questions or interventions, which may be provided to the end user for a response. The end user may then answer these new questions or interventions, beginning a new round of updating.

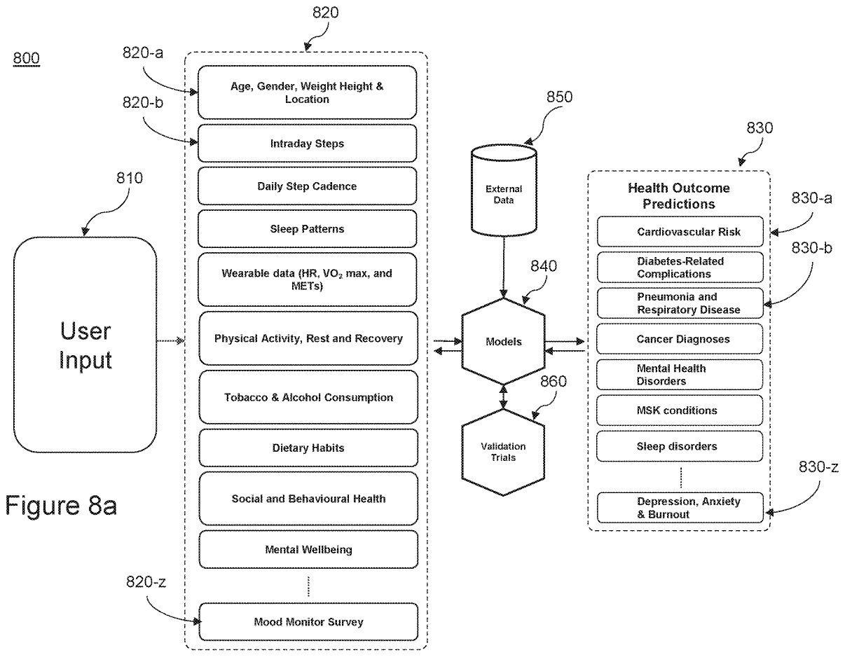

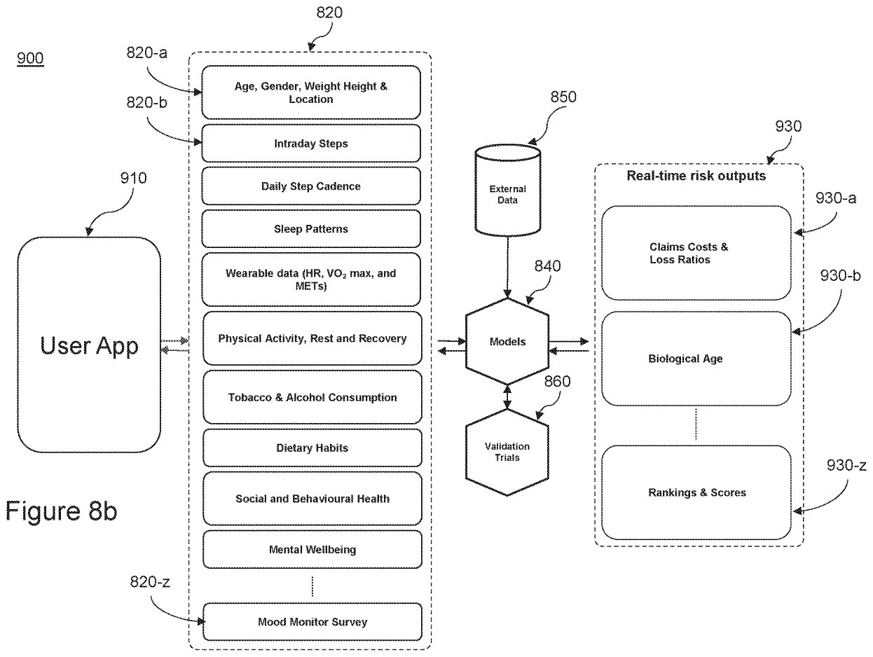

FIG. 8a shows a schematic diagram of a system 800 for managing a user's health. System 800 comprises a user health profile 820, a user risk profile 830, models 840, external data 850 and validation trials 860.

As shown in FIG. 8a, the user health profile 820 is comprised of a plurality of health data metrics 820a-z. FIG. 8a shows exemplar health data metrics 820a-z but the skilled person will understand that the term ‘health data metric’ encompasses any data which may impact a user's health (e.g. make a user more/less susceptible to a disease or illness or directly indicate good/poor health). The term ‘metrics’, as used herein, may equally refer to statistics, facts or information and may each comprise a single figure/value or a collection of values or trends. It will be understood that said health data metrics therefore describe a broad range of available metrics including, but not limited to: Age, Gender, Weight, Height, Location (e.g. ambient climate or nationality), Step Counters/Activity Trackers (e.g. Intraday Steps, Daily Step Cadence, Altitude Gain, Sleep Patterns), Wearable monitoring/data (e.g. Heart rate [HR], maximum bodily oxygen absorption [VO₂ max], metabolic equivalents [METs]), physical activity, rest recovery, dietary statistics/habits (e.g. tobacco consumption, alcohol consumption, calorie consumption), social or behavioural health (e.g. relationship status), mental health (e.g. mood monitor surveys, emotional wellbeing surveys).

The user health profile 820 is the collation of the plurality of health data metrics 820a-z, which may be grouped together on a memory or storage device of the system 800, as described herein. As shown in FIG. 8a, the user health profile 820 is updated by user input 810 to thereby create, amend or delete one or more health data metrics 820a-z. Typically a user provides an input via a user device, for example, via a mobile or computer device. In some embodiments, an application is provided on said mobile or computer device and configured to allow a user to input information/answer questions from system 800. As described above user input typically comprises an answer to a question but may, additionally or alternatively comprise inputting one or more of the above health data metrics when prompted. For example, a user may be presented with (e.g. displayed or shown) a question and a corresponding group of multiple-choice answer options, to which a user may click (or touch in embodiments wherein the device has a touchscreen) an answer to provide user input 810. Said user input 810 is transmitted to the user health profile 820 and is used to update (e.g. overwrite, append or delete information in the user health profile 820) one or more of the plurality of health data metrics 820a-z. Alternatively, the user health profile and user risk profile can be referred to as a dual user health profile.

In some embodiments, the user input 810 may cause the creation of one or more ‘new’ health data metrics 820a-z and/or the deletion of one or more health data metrics 820a-z from the user health profile 820, such that the number of health data metrics 820a-z is not fixed. It will be understood that as the user health profile 820 is updated by the input 810 of a specific user and that the specific health data metrics 820a-z present (e.g. extant) within the user health profile 820 vary based on said input 810 of a specific user, that therefore the user health profile is unique to a specific user. In some embodiments, a unique user health profile identifier (“profile ID”) may be associated with a user health profile 820 to thereby allow a specific instance of the user health profile 820 associated with a specific user to be identified and transmitted to within system 800.

FIG. 8a shows the models 840 transmitting and receiving data from and to each of the user health profile 820, user risk profile 830 and validation trial 860 and further shows the models 840 receiving external data 850 (e.g. from an external data source). Therefore, as described herein, the models 840 function as ‘nexus’ wherein data from each of the above elements may be combined and/or operated on. In particular, the models 840 may transmit an output to one or both of the user health profile 820 and the user risk profile 830 (e.g. for updating said user health profile 820 and/or user risk profile 830). Specifically, the models 840 allow data received from at least the user health profile 820 to be used to update the user risk profile 830, and vice versa. Preferably, updating of either the user health profile 820 or user risk profile 830 is based additionally, on the external data 850 and validation trial 860.

As shown in FIG. 8a, the user risk profile 830 is comprised of a plurality of risk metrics 830a-z (alternatively referred to as ‘health outcome predictions’). FIG. 8a shows a number of exemplar risk metrics 830a-z. Each risk metric 830a-z comprises data relating to a user's probability of contracting a specific condition (or ‘health outcome’), or a collection of specific conditions. FIG. 8a shows exemplar risk metrics 830a-z relating to specific conditions including: cardiovascular risk, diabetes related complications, pneumonia and respiratory disease, cancer diagnoses, mental health disorders, musculo-skeletal (“MSK”) conditions, sleep disorders, depression, anxiety and burnout. However, it will be understood that the term ‘condition’ as used herein includes, but is not limited to: an illness, disease, infection, malaise, disorder, ailment or application or other similar medical/health based outcome that the skilled person would recognise as relevant to a user's wellbeing.

Generally, the models 840 receive at least one health data metric 820a-z from the user health profile 820 and at least one risk metric 830a-z, from the user risk profile 830, and then determines, based on a given set of health data metrics 820a-z, the user's probability (or ‘risk’) of contracting a health outcome associated with the corresponding risk metric 830a-z. For example, the models 840 may receive health data metrics 820a-z corresponding to a heart rate of a user and an age of said user and risk metrics 830a-z corresponding to a cardiovascular risk and a diabetes risk. The models 840 may then determine a probability, given the provided health data metrics (e.g. given the users age and heart rate), of their current risk of cardiovascular outcomes/complications (e.g. heart attacks, arrythmia). In some embodiments, as described herein, the model can weight health data metrics based on their relevance to specific risk metrics 830a-z. In some embodiments the data relating to a user's probability of contracting a condition is represented as a probability density function (“PDF”), preferably a normalized PDF.

It will be understood that the use of separate user health profiles 820 and user risk profiles 830 allows more efficient data collection for user health risk management and results in improved health outcomes for a user on average. In conventional systems, typically data is collected to test for a specific risk/health outcome. For example, a patient (user) is identified as being over a certain age, statistically making them at higher risk of hypertension (high blood pressure), and therefore they are prompted to provide a blood pressure reading which can then be used to update their risk of hypertension, such that for each health outcome a risk of contracting said outcome must be assessed individually and only by analysing known symptoms/signs of elevated risk to a particular health outcome. In contrast, the present system 800 centralises the collating, and storing in a database, of health data into a user health profile 820, and then allows analysis of unlimited combinations of said health metrics against a plurality of risk metrics (outcomes) such that unconventional warning signs of adverse health outcomes that suggest a user is at risk of a particular disease, for example, can be identified. Furthermore, said risks of each health outcome can be assessed simultaneously and continuously/immediately, upon the user health profile being updated by the user input 810 and/or eternal data 850.

External data 850 is any data which is not provided to the models 840 by through user input 810. Typically, the external data 850 will comprise population statistical data and/or mapping of variables obtained from large-scale longitudinal medical datasets (e.g. national health registries/health services, national statistical surveys, cohort studies that pair long-term lifestyle data with subsequent health outcomes) to user profiles), i.e. data which is impersonal/unspecific to a user but instead relates to mean values of medical data for a population. However, in some embodiments, the external data 850 may comprise data unique to a user but which is not directly provided by the user. For example, a user may grant a permission to the models 840 to access their personal medical record, held by a third party (e.g. a medical insurer or private/public medical practice). It will be understood that this may allow the models 840 to populate (‘fill-in’) and/or update one or more of the health data metrics 820a-z without user input 810, thereby reducing the amount of user input 810 required by system 800, increasing user convenience and efficiency when interacting with system 800.

As described above, the user health profile 820 and thereby the system 800, is unique to a specific user. Therefore, in some embodiments the system 800 performs an initialisation step before proceeding to further functionality. Typically, external data 850 is used to update (e.g. provide initial data for) the user health profile 820 and user risk profile 830 such that each is populated with a plurality of health data metrics 820a-z and risk metrics 830a-z, respectively. However, in alternative embodiments, the user health profile 820 and user risk profile 830 are left unpopulated (i.e. an empty set), and population of health data metrics 820a-z occurs upon a first user input 810 being received. In said embodiments, population of the risk metrics 830a-z (of the user risk profile 830) may be performed by the models 840 upon receiving data from the user health profile 830 (after said first user input 810). Alternatively, or optionally, the models 840 may populate the user risk profile 830 based on receiving the external data 850, and/or based on a combination of the external data and the data received form the user health profile.

Generally, the user health profile 820 and user risk profile 830 are collections/collations of data metrics, as used herein, also described as databases.

Within system 800, the databases are typically stored digitally in a remote storage (e.g. remote from the location of user input 810, for example on a user device, and/or remote from a source of external data 850). In some embodiments, the databases are stored on a remote server, preferably a cloud server. However, the skilled person will understand that in some embodiments a copy of the databases may be stored on a user device, or transmitted to a third party (e.g. a source of the external data). Said ‘local’ copy of the databases may be used to facilitate offline use of system 800 (i.e. when disconnected from an internet connection) wherein the local copy of the user health profile 820, may be updated based on a received user input 810, and said updates synchronised with a remote (‘global’) copy when the system 800 next connects to the internet.

FIG. 8a shows the validation trials 860 transmitting and receiving data from the models 840. The validation trials may fine-tune the models based on a collection (or ‘set’) of validation data. Additionally or alternatively, the validation trials are configured to compare received data (i.e. from the models) to the validation data set to determine an accuracy of outputs or predictions received from the models 840. The received data is, typically, a determination made by the models 840 of at least one of the risk metrics 830a-z based on health data metrics 820a-z. It will be understood that the validation data set represents a ‘ground truth’ value to check or corroborate a determination of the models 840 and thus that the validation trials 860 allow an accuracy of the determination made by the model 840 to be obtained/made. Therefore, the validation trials 860 may also allow the models 840 to be fine-tuned/corrected prior to use or continually during use by a user. Beneficially, iteratively fine tuning and/or validating the model using the validation trials 860 allows the increasing corpus of DHQ data received by the described systems and methods to be incorporated into the model 840 creating an increasingly sophisticated model 840 over time.

The skilled person will understand that the validation trials 860 detecting a difference in model predicted/determined output and ground truth output does not necessarily mean the models 840 have made an incorrect determination, but rather that a learned correlation (i.e. between a specific health data metric 820a-z and a specific risk metric 830a-z) may need to be further tweaked in validation, perhaps in light of more rigorous provision of questions or in response to additional questions being added. Beneficially, applying a previously unseen dataset to the model 840 in the validation trials 860 allows the model parameters to be tested on and tweaked based on new but verified data, Advantageously, therefore said specific health data metric 820a-z may novelly be used to evaluate said specific risk metric 830a-z. The validation trials 860 provides accuracy and output credibility to the models 840 by auditing the certainty of output response and in some cases finetuning the models 840 to account for low certainty outcomes. In some examples, validation trials 860 may be additionally conducted in a peer-reviewed fashion adhering a rigour similar to that of standard medical trials. For example, a sample population of 1400+ volunteers provided data, and the models used to predict their health outcomes and validate how accurate the model was.

In another example, it may be known that age and alcohol consumption contribute to an elevated risk of cardiovascular disease, models 840 may also determine the risk metric of cardiovascular disease based on a health metric of sleep duration.

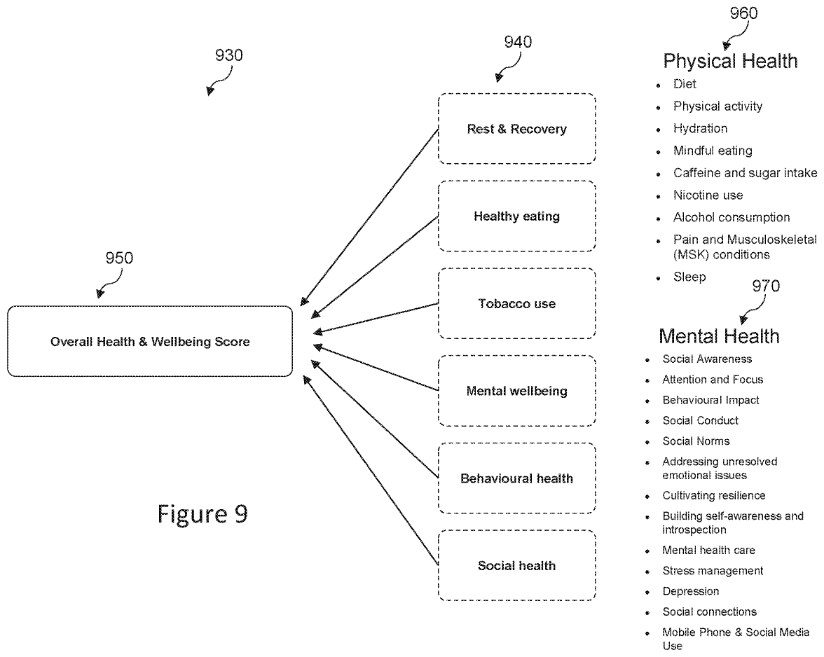

FIG. 8b shows system 900, similar to system 800, where like numerals have been used for like features. FIG. 9b shows an alternative embodiment of system 800, wherein the user input is provided by a user application (an “app”) 910. FIG. 8b further shows the user risk profile 830 of system 800, implemented as risk output interface 930, specifically a real-time risk output interface.

The user application 910 allows a user to have two-way engagement with the user health profile 820, and via the models 840 with the risk output interface 930. It will be understood that two-way engagement may refer to the user health profile 820 being configured to both receive user input data from the user application 910 and transmit information/feedback to the user application 910. It will be understood that this may allow the user application to display one or more of the plurality of health data metrics 820a-z to the user, or a summary thereof. Hence the user may be able to verify the accuracy of (the health data metrics 820a-z comprising) the user health profile 820 and/or be displayed notifications as to missing or otherwise faulty data, to thereby prompt a user to rectify the missing/faulty data by providing further input. It will also be understood that a user may be prompted to alter their physical actions, measured by the health data metrics 820a-z based on reviewing said health data metrics 820a-z to thereby improve a health outcome of a user.

Furthermore, FIG. 8b shows the implementation of the risk output interface 930. The risk output interface 930 provides an additional layer of analysis (herein referred to as meta-metrics 930a-z) to the user risk profile 830, and thereby allows the underlying risk metrics 830a-z to be more easily understood to a user or to a third party accessing the system 900. FIG. 9b shows exemplar meta-metrics 930a-z including: claim costs, loss ratios, biological age, (health) rankings & scores, however, the skilled person will understand that a plurality of alternative or additional meta-metrics 930a-z could be determined. The meta-metrics are conditional weighted combinations of risk metrics 830a-z to thereby summarise relevant health information to a user or third party. For example, a biological age 930-b may combine risk metrics 930a-z associated with a risk of stroke and risk of hypertension and compare these values (or an average of these values) to a statistical population average (e.g., provided by external data 850), to thereby indicate to a user how the function of their body compares to other users of a similar age. It will be understood that this may be more easily accessible, and simpler to understand, to a user than simply listing the underlying health metrics. Similarly, the claim cost meta-metric 930a may provide a third party (e.g. an insurer) a means to rapidly assess a user's overall health for the purposes of providing appropriate insurance levels or underwriting. In system 900, the updating of risk metrics 830a-z in FIG. 8a, still occurs and as such the models 840 may receive one or both of the underlying risk metrics 830a-z or the meta-metrics 930a-z from the user risk profile 930, dependent on the analysis required and/or the query by a user application or external third party.

FIG. 9 shows an aspect of a health profile to inform the dynamic health questionnaires as a plurality of meta-metrics 930a-z as described in respect of FIG. 8b, specifically an overall health meta-metric 930, FIG. 9 further showing the plurality of component risk metrics 940 and the meta-metric value 950 (“overall health & wellbeing score”). Lists 960, 970 describe additional or alternative user health metrics which may in other embodiments be components of the overall health meta-metric 930. In some embodiments, the overall health meta-metric 930 is comprised entirely of risk metric components; however, it will be understood that typically said meta-metrics are comprised of a conditional combination of both health data metrics and risk metrics. It will be understood, further, that the models 840, described in respect of FIGS. 8a and 8b, are configured to combine a plurality of risk metrics and/or health data metrics to output the overall health meta-metric 1050, which may be stored in the user risk profile 830. In some embodiments, wherein the meta-metric is a PDF, the overall health and wellbeing score 950 may be displayed to the user graphically. In some embodiments, historical values for said meta-metrics 930a-z are stored in the user risk profile 930 such that the overall health and wellbeing score 950 may be displayed as a trend over time, or as a difference to a previous score.

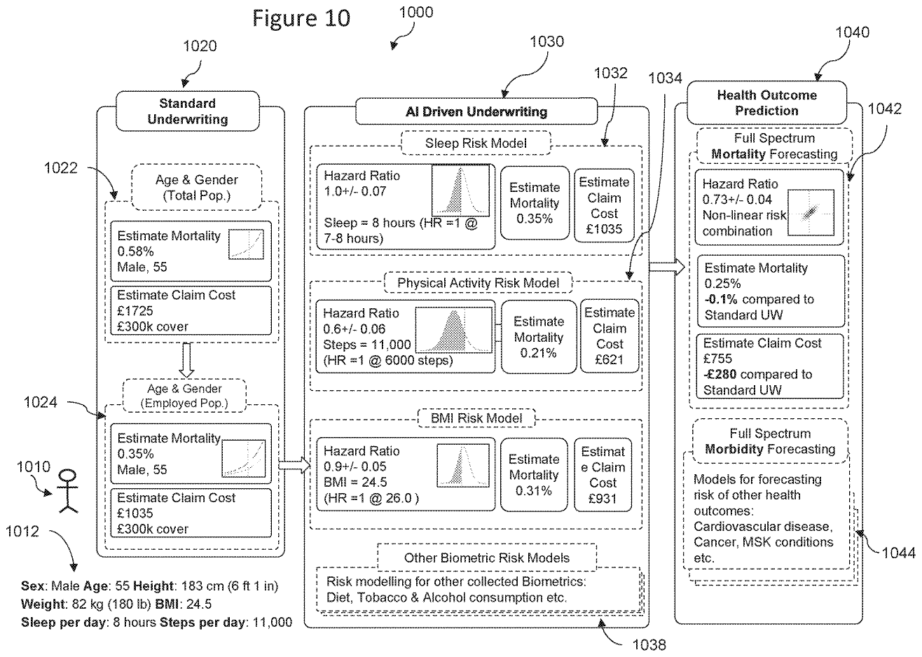

FIG. 10 shows a schematic of the process steps used by the models 840 to determine a risk metric 830. FIG. 10 shows a user 1010, with a user health profile comprising a plurality of health data metrics 1012 as shown, including: sex, age, height, weight, body mass index (BMI), sleep per day and steps per day of the user 1010. One or more of the health data metrics 1012 may be processed by standard underwriting means 1020, for example, as shown, by comparison to a mean mortality of a population 1022, or of the employed population 1024. One or more other health data metrics 1012 may be processed via AI driven underwriting 1030, using models (similar to the models 840 of FIGS. 8a & 8b). For example, the sleep per day health data metric 1012 is processed by a sleep risk model while the steps per day health data metric 1012 is processed by a physical activity model. The AI driven modelling/underwriting may be further based on one or other biometric risk models 1038 and furthermore may be based on an output of the standard underwriting 1020 processes. The AI driven underwriting outputs a health prediction outcome 1040 (alternatively referred to as a ‘risk metric’) corresponding to a specific health outcome. FIG. 10 shows for example mortality risk metric 1042, thereby providing a prediction of the user's 1010 probability of death. The AI driven underwriting can provide further associated risk metric forecast 1044 using one or more forecasting models to predict a trend in the associated risk metric over a future timespan.

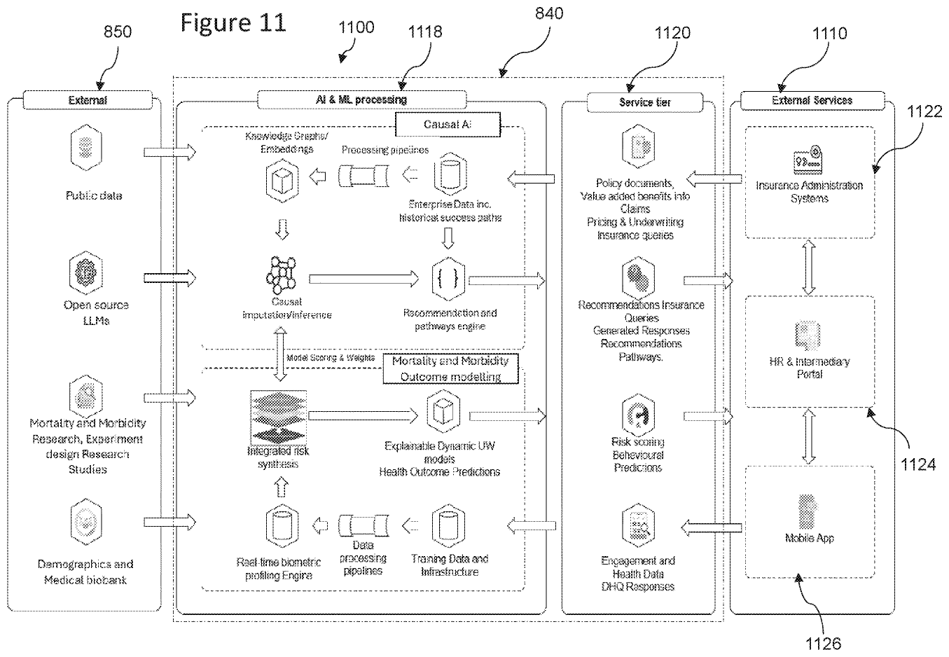

FIG. 11 shows a schematic of the interaction of system 1100 with external services, similar to the system 800,900 of FIG. 8-9, where like numerals have been used for like references. FIG. 11, shows the system 1100 comprising models 840, receiving external data 850 from external data sources. The models 840 further comprise an analysis module 1118, and a service tier 1120, the service tier being configured to engage with one or more external services 1110. Specifically shown in FIG. 11 are external services including insurance administration systems 1122, human resources (HR) and intermediary (business) portals 1124, and a mobile app 1126. As shown in FIG. 11, the service tier 1120 is specifically configured to receive, for example, policy documents from an external service, such as an insurance administration system 1122. It will be understood that the service tier may have various sub-components for communicating information to and from said external services 1110, for example, a library of various application programming interfaces (APIs), which are known in the art.

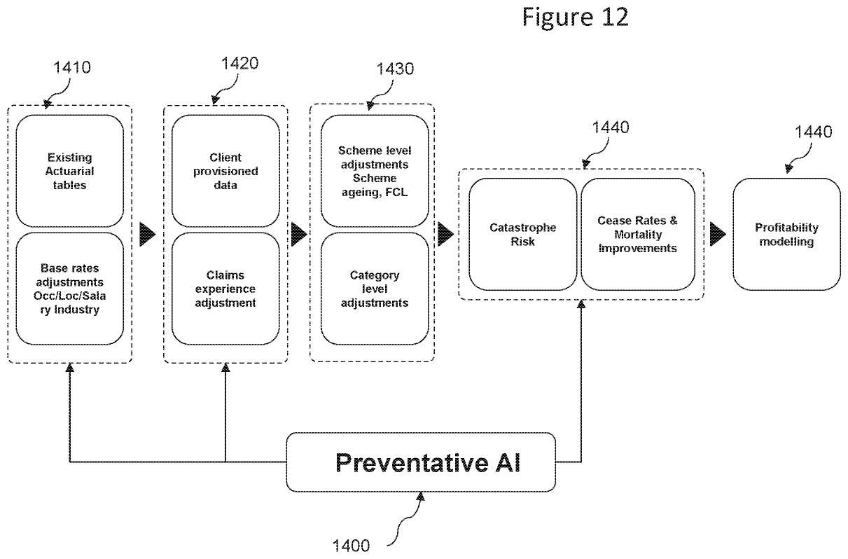

FIG. 12 shows a schematic diagram of the implementation of the AI driven modelling 1400 and/or underwriting of FIG. 10 according to an iterative embodiment. In FIG. 12, AI driven modelling 1400 may be applied repeatedly during the determination of risk metrics 1440, shown specifically as catastrophe risk and cease rates. AI driven modelling may be applied at any or all of: the initial base weighting stage 1410; during initial user (client) input 1420; during scheme level adjustments 1430; or during the calculation of risk metrics 1440 themselves. FIG. 12 further shows that said risk metrics may then be computed into profitability modelling system 1440.

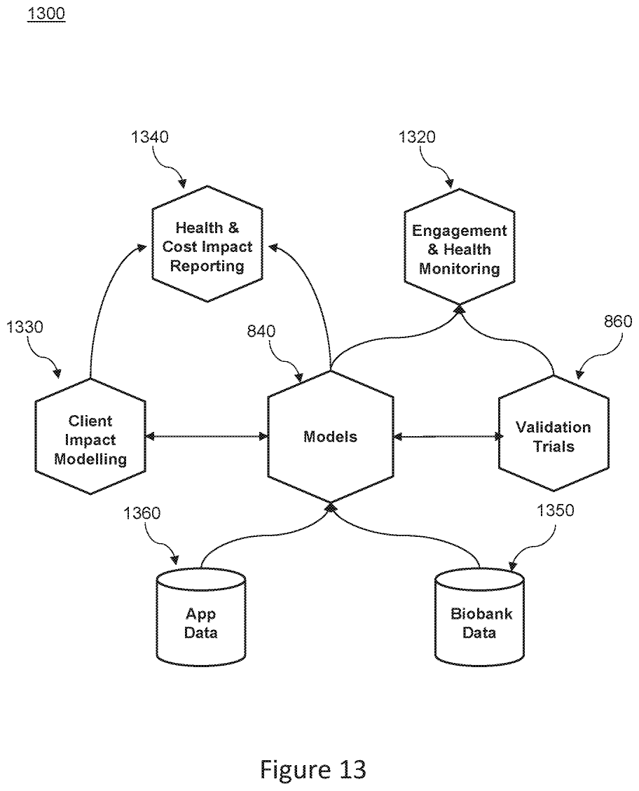

FIG. 13 shows a schematic diagram of a system 1300, similar to the system of system 800 and 900 of FIGS. 8a and 8b, where like numerals have been used for like references. FIG. 13 further shows the external data decomposed into a medical dataset 1350 and app data 1360, and the system 1300 comprising: an engagement and health monitoring module 1320, a health & cost impact reporting module 1340 and a client impact modelling module 1330. Said modules allow the system 1300 to provide diagnostic tools to a user. Furthermore, the validation trials 860 are shown transmitting data to the engagement & health monitoring module.

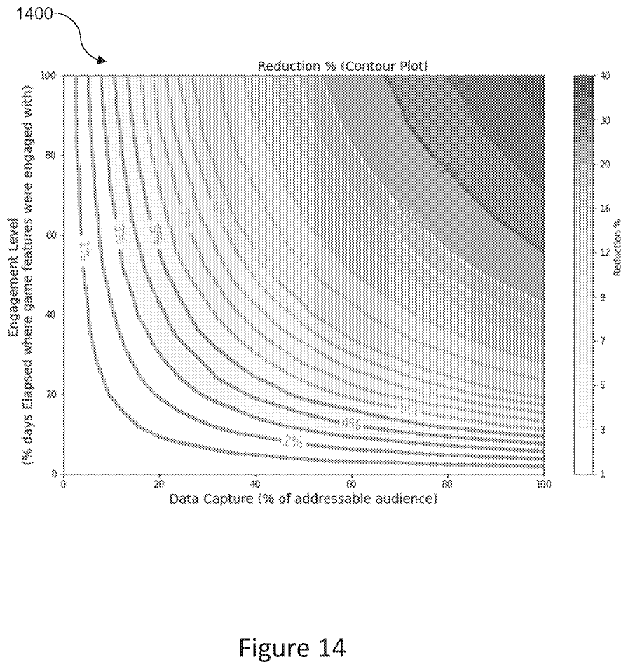

FIG. 14 shows a contour plot, demonstrating a relationship between reduction of risk for end users and/or a 3rd-party client 160 responsible for providing insurance and engagement with the system. Percentages shown in the contour plot denote the reduction of risk as a function of the determined engagement level or metric, in this instance being the percentage of days elapsed where the system, which in this example additionally comprises game features, and the percentage of potential end users (i.e. data capture) who engage with the disclosed system. As the percentage of the end users who engage with the system for longer increases, then the corresponding risk to an end user and/or a 3rd-party client 160 responsible for providing insurance decreases.

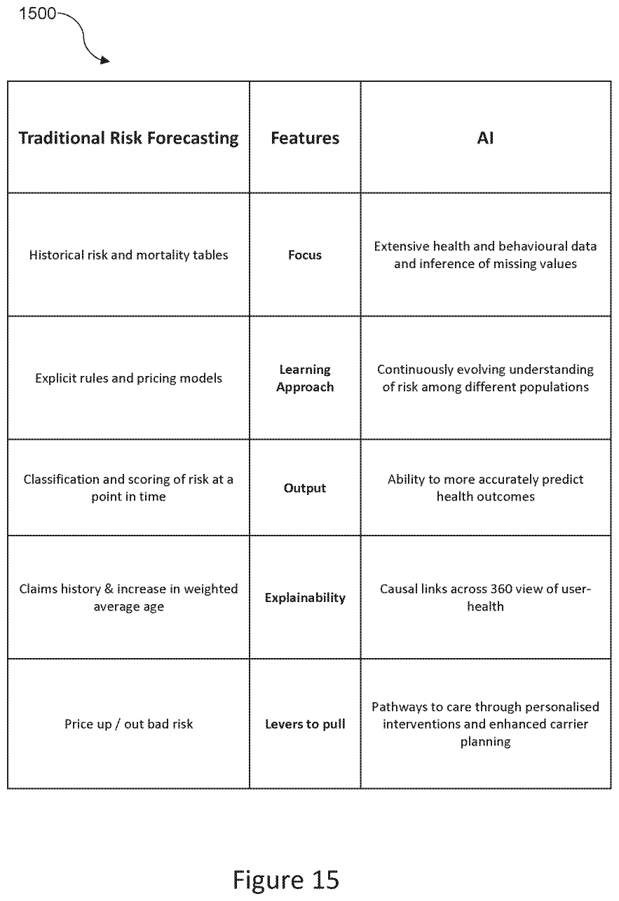

FIG. 15 shows a table showing key differences between traditional risk forecasting approaches, which work using population-level metrics for health and risk, with an approach incorporating AI-driven analysis and forecasting. For instance, an AI-driven approach may focus on leveraging extensive health and behavioural data and inference of missing values, compared to historical risk and mortality tables. As a further example, an AI-driven approach may utilise continuously evolving understanding of risk among different populations, compared to explicit rules and pricing models. As a further example, an AI-driven approach may more accurately predict health outcomes, compared to classification and scoring of risk occurring for present values. As a yet further example, an AI-driven approach may allow for causal links across a “360 view” of user health, compared to claims history and an increase in weighted average age. An AI-driven approach may also provide many pathways to care through personalised interventions and enhanced carrier planning, compared to adjusting pricing for insurance premiums for poorly appraised risk outcomes

It will be understood that the present invention has been described above purely by way of example, and modifications of detail can be made within the scope of the invention.

Reference numerals appearing in the claims are by way of illustration only and shall have no limiting effect on the scope of the claims.

What is claimed is:

1. A method of updating a user risk profile for controlling a user health outcome, the method comprising:
receiving a user health profile, comprising a plurality of health data metrics;
receiving a user risk profile, comprising a plurality of risk metrics;
receiving historical outcome data comprising correlated historical health data metrics and historical risk metrics; and
generating, based on the user health profile, a subset of the plurality of health data metrics from the user health profile;
determining, based on the historical outcome data, a correlation between any of the subset health metrics; and
updating, based on a determined correlation, a subset of the plurality of risk metrics.

2. The method of claim 1, the method further comprising updating all of the plurality of risk metrics of the user risk profile.

3. The method of claim 1, the method further comprising:
receiving external data, and
wherein determining a correlation is further based on the external data.

4. The method of claim 3, wherein the external data comprises health related population statistics.

5. The method of claim 1, the method further comprising:
validating, by a validation trial, the determination of a correlation, wherein the validation trial compares the determined correlation to pre-existing medical data; and
upon detecting a difference between the determined correlation and the preexisting medical data, raising an error.

6. The method of claim 1, the method further comprising:
generating, by interpolating between a first health data metric and a second health data metric, a third health data metric; and
appending the subset of the plurality of health data metrics with the third health data metric.

7. The method of claim 1, the method further comprising:
providing, to a third party, the updated user risk profile, preferably further providing the user health profile.

8. The method of claim 1, the method further comprising:
outputting the updated user risk profile to a user health system, wherein the user health system is configured to transmit the updated user risk profile to at least one of:
a third party;
a database;
a user interface, preferably on a user device.

9. A method of determining a health profile of a user for controlling a user health outcome, comprising:
retrieving a first question from a database of health questions;
providing the first question to a user interface;
receiving from the user interface an input from a user indicating a first answer to the first question;
updating the user health profile in dependence on the first answer; and
retrieving a second question from the database;
wherein retrieving the second question is dependent on a historical correlation between the second question and the first answer.

10. The method of claim 9, wherein each question in the database of health questions is associated with at least one of a plurality of health topics, wherein the first and second question are associated with different health topics.

11. The method of claim 9, wherein retrieving the first and second question is performed by a machine learning model trained to determine a correlation between the second question and the first answer to the first question.

12. The method of claim 9, wherein retrieving the second question from the database is in further dependence on the updated user health profile.

13. The method of claim 9, the method further comprising:
providing the second question to a user interface;
receiving from the user interface an input from a user indicating a second answer to the second question;
updating the user health profile in dependence on the second answer; and
retrieving a third question from the database;
wherein retrieving the third question is dependent on a historical correlation between the third question and the second answer,
preferably wherein the method is performed iteratively.

14. The method of claim 9, wherein user answers to each provided question are stored in a database as historical response data; and wherein retrieving the second question from the database is in further dependence on the historical response data.

15. The method of claim 9, wherein the user health profile comprises a risk score defining a probability of a given health outcome, wherein retrieving the second question from the database is in further dependence on the risk score.

16. The method of claim 9, wherein each question in the database of health questions is associated with a base weight value, wherein retrieving the second question from the database is in further dependence on a probability distribution calculated from the base weight values.

17. The method of claim 16, wherein the base weight value is multiplied by a seasonality factor of the question, wherein each question the database is associated with a date and the seasonality factor is based on a difference between a current date and the associated date.

18. The method of claim 9, wherein each question in the database of questions is associated with an eligibility value and wherein the method is configured to only retrieve a question with an eligibility value above a predetermined eligibility threshold.

19. The method of claim 18, wherein the eligibility values of each question in the database of health questions are calculated based on a plurality of trigger conditions.

20. The method of claim 9, wherein the user health profile is a first user health profile, the method further comprising a second user health profile.
